(19) United States
(12) Patent Application Publication
(10) Pub. No.: US 2008/0262040 A1
(43) Pub. Date: Oct. 23, 2008

(54) Chemical Compounds

(75) Inventors: James Francis Callahan, King of Prussia, PA (US); Jeffrey K. Kerns, King of Prussia, PA (US); Xichen Lin, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SMITHKLINE BEECHAM CORPORATION

(21) Appl. No.: 12/091,491

(22) PCT Filed: Oct. 20, 2006
(86) PCT No.: PCT/US06/60098

§ 371 Date: Apr. 25, 2008

Related U.S. Application Data
(60) Provisional application No. 60/729,969, filed on Oct. 25, 2005.

(51) Int. Cl. A61K 31/4535; C07D 211/06; A61P 25/28; A61P 3/10; A61P 31/18; A61P 11/06

(52) U.S. Cl. 514/322; 546/199

(57) ABSTRACT

The invention is directed to novel indazole carboxamide derivatives. Specifically, the invention is directed to compounds according to formula (I):

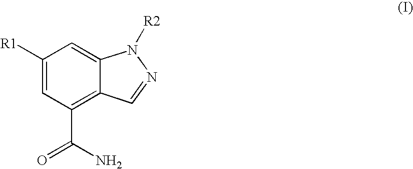

where R1 and R2 are as defined below. These compounds are useful in the treatment of disorders associated with inappropriate IKK2 (also known as IKKβ) activity, in particular in the treatment and prevention of disorders mediated by IKK2 mechanisms including inflammatory and tissue repair disorders. Such disorders include rheumatoid arthritis, asthma, and COPD (chronic obstructive pulmonary disease).

### FIELD OF THE INVENTION

The invention is directed to certain indazole carboxamide compounds, which are inhibitors of kinase activity. More specifically, the compounds are IKK2 inhibitors. These compounds are useful in the treatment of disorders associated with inappropriate IKK2 (also known as IKKβ) activity, in particular in the treatment and prevention of disorders mediated by IKK2 mechanisms including inflammatory and tissue repair disorders. Such disorders include rheumatoid arthritis, asthma, and COPD (chronic obstructive pulmonary disease).

### BACKGROUND OF THE INVENTION

An important large family of enzymes is the protein kinase enzyme family. Currently, there are about 500 different known protein kinases. However, because three to four percent of the human genome is a code for the formation of protein kinases, there may be many thousands of distinct and separate kinases in the human body. Protein kinases serve to catalyze the phosphorylation of an amino acid side chain in various proteins by the transfer of the γ-phosphate of the ATP-Mg²⁺ complex to said amino acid side chain. These enzymes control the majority of the signaling processes inside cells, thereby governing cell function, growth, differentiation and destruction (apoptosis) through reversible phosphorylation of the hydroxyl groups of serine, threonine and tyrosine residues in proteins. Studies have shown that protein kinases are key regulators of many cell functions, including signal transduction, transcriptional regulation, cell motility, and cell division. Several oncogenes have also been shown to encode protein kinases, suggesting that kinases play a role in oncogenesis. These processes are highly regulated, often by complex intermeshed pathways where each kinase will itself be regulated by one or more kinases. Consequently, aberrant or inappropriate protein kinase activity can contribute to the rise of disease states associated with such aberrant kinase activity. Due to their physiological relevance, variety and ubiquitousness, protein kinases have become one of the most important and widely studied family of enzymes in biochemical and medical research.

The protein kinase family of enzymes is typically classified into two main subfamilies: Protein Tyrosine Kinases and Protein Serine/Threonine Kinases, based on the amino acid residue they phosphorylate. The serine/threonine kinases (PSTK), includes cyclic AMP- and cyclic GMP-dependent protein kinases, calcium and phospholipid dependent protein kinase, calcium- and calmodulin-dependent protein kinases, casein kinases, cell division cycle protein kinases and others. These kinases are usually cytoplasmic or associated with the particulate fractions of cells, possibly by anchoring proteins. Aberrant protein serine/threonine kinase activity has been implicated or is suspected in a number of pathologies such as rheumatoid arthritis, psoriasis, septic shock, bone loss, many cancers and other proliferative diseases. Accordingly, serine/threonine kinases and the signal transduction pathways which they are part of are important targets for drug design. The tyrosine kinases phosphorylate tyrosine residues. Tyrosine kinases play an equally important role in cell regulation. These kinases include several receptors for molecules such as growth factors and hormones, including epidermal growth factor receptor, insulin receptor, platelet derived growth factor receptor and others. Studies have indicated that many tyrosine kinases are transmembrane proteins with their receptor domains located on the outside of the cell and their kinase domains on the inside. Much work is also under progress to identify modulators of tyrosine kinases as well.

Nuclear factor κB (NF-κB) belongs to a family of closely related dimeric transcription factor complexes composed of various combinations of the Rel/NF-κB family of polypeptides. The family consists of five individual gene products in mammals, RelA (p65), NF-κB1 (p50/p105), NF-κB2 (p49/p100), c-Rel, and RelB, all of which can form hetero- or homodimers. These proteins share a highly homologous 300 amino acid “Rel homology domain” which contains the DNA binding and dimerization domains. At the extreme C-terminus of the Rel homology domain is a nuclear translocation sequence important in the transport of NF-κB from the cytoplasm to the nucleus. In addition, p65 and cRel possess potent transactivation domains at their C-terminal ends.

The activity of NF-κB is regulated by its interaction with a member of the inhibitor IκB family of proteins. This interaction effectively blocks the nuclear localization sequence on the NF-κB proteins, thus preventing migration of the dimer to the nucleus. A wide variety of stimuli activate NF-κB through what are likely to be multiple signal transduction pathways. Included are bacterial products (LPS), some viruses (HIV-1, HTLV-1), inflammatory cytokines (TNFα, IL-1), environmental and oxidative stress and DNA damaging agents. Apparently common to all stimuli however, is the phosphorylation and subsequent degradation of IκB. IκB is phosphorylated on two N-terminal serines by the recently identified IκB kinases (IKK-α and IKK-β). IKK-β is also known as IKK2. Site-directed mutagenesis studies indicate that these phosphorylations are critical for the subsequent activation of NF-κB in that once phosphorylated the protein is flagged for degradation via the ubiquitin-proteasome pathway. Free from IκB, the active NF-κB complexes are able to translocate to the nucleus where they bind in a selective manner to preferred gene-specific enhancer sequences. Included in the genes regulated by NF-κB are a number of cytokines and chemokines, cell adhesion molecules, acute phase proteins, immunoregualtory proteins, eicosanoid metabolizing enzymes and anti-apoptotic genes.

It is well-known that NF-κB plays a key role in the regulated expression of a large number of pro-inflammatory mediators including cytokines such as TNF, IL-1β, IL-6 and IL-8, cell adhesion molecules, such as ICAM and VCAM, and inducible nitric oxide synthase (iNOS). Such mediators are known to play a role in the recruitment of leukocytes at sites of inflammation and in the case of iNOS, may lead to organ destruction in some inflammatory and autoimmune diseases.

The importance of NF-κB in inflammatory disorders is further strengthened by studies of airway inflammation including asthma, in which NF-κB has been shown to be activated. This activation may underlie the increased cytokine production and leukocyte infiltration characteristic of these disorders. In addition, inhaled steroids are known to reduce airway hyperresponsiveness and suppress the inflammatory response in asthmatic airways. In light of the recent findings with regard to glucocorticoid inhibition of NF-κB, one may speculate that these effects are mediated through an inhibition of NF-κB.

Further evidence for a role of NF-κB in inflammatory disorders comes from studies of rheumatoid synovium. Although NF-κB is normally present as an inactive cytoplasmic complex, recent immunohistochemical studies have indicated that NF-κB is present in the nuclei, and hence active, in the cells comprising rheumatoid synovium. Furthermore, NF-κB has been shown to be activated in human synovial cells in response to stimulation with TNF-α or IL-1β. Such a distribution may be the underlying mechanism for the increased cytokine and eicosanoid production characteristic of this tissue. See Roshak, A. K., et al., J. Biol. Chem., 271, 31496-31501 (1996). Expression of IKK-β has been shown in synoviocytes of rheumatoid arthritis patients and gene transfer studies have demonstrated the central role of IKK-β in stimulated inflammatory mediator production in these cells. See Aupperele et al. J. Immunology 1999. 163:427-433 and Aupperle et al. J. Immunology 2001; 166:2705-11. More recently, the intra-articular administration of a wild type IKK-β adenoviral construct was shown to cause paw swelling while intra-articular administration of dominant-negative IKKβ inhibited adjuvant-induced arthritis in rat. See Tak et al. Arthritis and Rheumatism 2001, 44:1897-1907.

The NF-κB/Rel and IκB proteins are also likely to play a key role in neoplastic transformation and metastasis. Family members are associated with cell transformation in vitro and in vivo as a result of over expression, gene amplification, gene rearrangements or translocations. In addition, rearrangement and/or amplification of the genes encoding these proteins are seen in 20-25% of certain human lymphoid tumors. Further, NF-κB is activated by oncogenic ras, the most common defect in human tumors and blockade of NF-κB activation inhibits ras mediated cell transformation. In addition, a role for NF-κB in the regulation of apoptosis has been reported strengthening the role of this transcription factor in the regulation of tumor cell proliferation. TNF, ionizing radiation and DNA damaging agents have all been shown to activate NF-κB which in turn leads to the upregulated expression of several anti-apoptotic proteins. Conversely, inhibition of NF-κB has been shown to enhance apoptotic-killing by these agents in several tumor cell types. As this likely represents a major mechanism of tumor cell resistance to chemotherapy, inhibitors of NF-κB activation may be useful chemotherapeutic agents as either single agents or adjunct therapy. Recent reports have implicated NF-κB as an inhibitor of skeletal cell differentiation as well as a regulator of cytokine-induced muscle wasting (Guttridge et al. Science; 2000; 289: 2363-2365.) further supporting the potential of NFκB inhibitors as novel cancer therapies.

Several NF-κB inhibitors are described in C. Wahl, et al. J. Clin. Invest. 101(5), 1163-1174 (1998), R. W. Sullivan, et al. J. Med. Chem. 41, 413-419 (1998), J. W. Pierce, et al. J. Biol. Chem. 272, 21096-21103 (1997).

The marine natural product hymenialdisine is known to inhibit NF-κB. Roshak, A., et al., JPET, 283, 955-961 (1997). Breton, J. J and Chabot-Fletcher, M. C., JPET, 282, 459-466 (1997).

Additionally, patent applications have been filed on aminothiophene inhibitors of the IKK2, see Callahan, et al., WO 2002030353; Baxter, et al., WO 2001058890, Faull, et al., WO 2003010158; Griffiths, et al., WO2003010163; Fancelli, et al., WO 200198290; imidazole inhibitors of IKK2, see Callahan, et al., WO 200230423; anilinophenylpyrimidine inhibitors of IKK2, see Kois, et al., WO 2002046171; β-carboline inhibitors of IKK2, see Ritzeler, et al, WO 2001068648, Ritzeler, et al, EP 1134221; Nielsch, et al. DE 19807993; Ritzeler, et al., EP 1209158; indole inhibitors of IKK2, see Ritzeler, et al., WO 2001030774; benzimidazole inhibitors of the IKK2, see Ritzeler, et al., DE 19928424; Ritzeler et al, WO 2001000610; aminopyridine inhibitors of IKK2, see Lowinger, et al, WO 2002024679; Murata, et al, WO 2002024693; Murata, et al., WO 2002044153;pyrazolaquinazoline inhibitors of IKK2, see Beaulieu, et al., WO 2002028860; Burke et al, WO 2002060386, Burke, et al. US 20030022898; quinoline inhibitors of IKK2, Browner, et al., WO2002041843, Browner, et al., US 20020161004 and pyridylcyanoguanidine inhibitors of IKK2, see Bjorkling, et al., WO 2002094813, Binderup et al, WO 2002094322 and Madsen, et al., WO 200294265.The natural products staurosporine, quercetin, K252a and K252b have been shown to be IKK2 inhibitors, see Peet, G. W. and Li, J. J. Biol. Chem., 274, 32655-32661 (1999) and Wisniewski, D., et al., Analytical Biochem. 274, 220-228 (1999). Synthetic inhibitors of IKK2 have also been described, see Burke, et al. J. Biol. Chem., 278, 1450-1456 (2003) and Murata, et al., Bioorg. Med. Chem. Lett., 13, 913-198 (2003) have described IKK2 inhibitors.

Thus, attempts have been made to prepare compounds that inhibit IKK2 activity and a number of such compounds have been disclosed in the art. However, in view of the number of pathological responses that are mediated by IKK2, there remains a continuing need for inhibitors of IKK2 which can be used in the treatment of a variety of conditions.

The present inventors have discovered novel indazole carboxamide compounds, which are inhibitors of kinase activity, in particular inappropriate IKK2 activity. Such indazole carboxamide derivatives are therefore useful in the treatment of disorders associated with inappropriate kinase, in particular inappropriate IKK2 activity in particular in the treatment and prevention of disease states mediated by IKK2 mechanisms including inflammatory and tissue repair disorders, particularly rheumatoid arthritis, inflammatory bowel disease, asthma and COPD (chronic obstructive pulmonary disease); osteoarthritis, osteoporosis and fibrotic diseases; dermatosis, including psoriasis, atopic dermatitis and ultraviolet radiation (UV)-induced skin damage; autoimmune diseases including systemic lupus eythematosus, multiple sclerosis, psoriatic arthritis, alkylosing spondylitis, tissue and organ rejection, Alzheimer's disease, stroke, atherosclerosis, restonosis, diabetes, glomerulonephritis, cancer, including Hodgkins disease, cachexia, inflammation associated with infection and certain viral infections, including acquired immune deficiency syndrome (AIDS), adult respiratory distress syndrome, and Ataxia Telangiestasia.

### SUMMARY OF THE INVENTION

The invention is directed to novel indazole carboxamide derivatives. Specifically, the invention is directed to compounds according to formula (I):

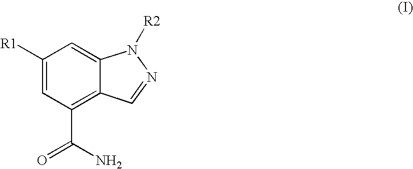

where R1 and R2 are as defined below.

### DETAILED DESCRIPTION OF THE INVENTION

The invention is directed to compounds according to formula (I):

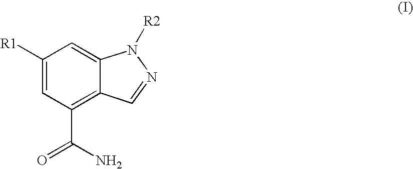

wherein:
R1 is optionally substituted aryl or optionally substituted heteroaryl,
where said aryl and heteroaryl are optionally substituted with one to three substituents each independently selected from the group consisting of: halo, optionally substituted C₁-C₆ alkyl, optionally substituted C₁-C₆ haloalkyl, —CN, —N(Rb)SO₂Re, —N(Rb)C(O)Ra, —C(O)NRaRb, —C(O)NRxRy, —SO₂NRaRb, —SO₂NRxRy, —ORc, —N(Rb)C(O)NRaRb, —N(Rb)C(O)NRxRy, and —N(Rb)C(O)ORd, where said C₁-C₆ alkyl and C₁-C₆ haloalkyl are optionally substituted with one to three substituents each independently selected from the group consisting of: NRaRb, —N(Rb)SO₂Re, C(O)Ra, —C(O)NRaRb, C₃-C₆ cycloalkyl, ORc, phenyl, and heterocycloalkyl optionally substituted with one or two C₁-C₆ alkyl groups;
   R2 is H, or the group —YZ;
   Y is a bond or C₁-C₆ alkylene;
   Z is C₃-C₆ cycloalkyl, aryl, heteroaryl, or heterocycloalkyl each of which is optionally substituted by one R3 group;

### R3 is R4, —S(O)₂R4, —C(O)R4, —C(O)OR4, —N(Rf)C(O)R4, —C(O)N(Rf)R4, —NHC(O)NHR4, —S(O)₂N(Rf)R4, or —N(Rf)S(O)₂R4;

R4 is optionally substituted C₁-C₆ alkyl, optionally substituted aryl, optionally substituted C₃-C₆ cycloalkyl, optionally substituted heteroaryl, or optionally substituted heterocycloalkyl,
wherein said C₁-C₆ alkyl is optionally substituted with one to three substituents each independently selected from the group consisting of: halo, —ORi, —NRgRh, —NHC(O)Rg, and Rj; and where said aryl and heteroaryl are optionally substituted by one to three substituents each independently selected from the group consisting of: halo, —ORg, nitro, cyano, —CF₃, C₁-C₆ alkyl, C(O)Rg, COORg, —NRgRh, —NHC(O)Rg, —C(O)NRgRh, —S(O)₂Rg, —NHS(O)₂Rg, and —S(O)₂NRgRh; and where said C₃-C₆ cycloalkyl and heterocycloalkyl are optionally substituted by one to three substituents each independently selected from the group consisting of: —OH, oxo, C₁-C₆ alkyl, and C₁-C₆ haloalkyl;
   each Ra is independently selected from the group consisting of: H, optionally substituted C₁-C₃ alkyl, optionally substituted phenyl, optionally substituted heteroaryl, optionally substituted C₃-C₇ cycloalkyl, and optionally substituted heterocycloalkyl, where said C₁-C₃ alkyl is optionally substituted with one to three substituents each independently selected from the group consisting of: halo, ORc, C₁-C₆ haloalkyl, phenyl, and heteroaryl; and where said phenyl, heteroaryl, C₃-C₇ cycloalkyl, and heterocycloalkyl are optionally substituted with one to three substituents each independently selected from the group consisting of: halo, ORc, C₁-C₆ alkyl, and C₁-C₆ haloalkyl;
   each Rb is independently selected from the group consisting of: H and optionally substituted C₁-C₃ alkyl, where said C₁-C₃ alkyl is optionally substituted with one to three ORc groups;
   each Rc is independently selected from the group consisting of: H, optionally substituted C₁-C₆ alkyl, optionally substituted C₁-C₆ haloalkyl, optionally substituted C₃-C₇ cycloalkyl, optionally substituted heterocycloalkyl, optionally substituted aryl, optionally and substituted heteroaryl, where said C₁-C₆ alkyl and C₁-C₆ haloalkyl are optionally substituted with one to three substituents each independently selected from the group consisting of: C₃-C₆ cycloalkyl, phenyl, heterocycloalkyl, and heteroaryl; and where said aryl and heteroaryl are optionally substituted with one to three substituents each independently selected from the group consisting of: halo, C₁-C₃ alkyl, C₁-C₃ haloalkyl and OH; and where said C₃-C₇ cycloalkyl and heterocycloalkyl are optionally substituted with one to three C₁-C₃ alkyl groups;
   each Rd is independently optionally substituted C₁-C₃ alkyl, where said C₁-C₃ alkyl is optionally substituted with one to three substituents each independently selected from the group consisting of: C₃-C₆ cycloalkyl; phenyl optionally substituted with one to three substituents each independently selected from the group consisting of: halo, C₁-C₆ alkyl, and C₃-C₆ cycloalkyl; and heteroaryl optionally substituted with one to three substituents each independently selected from the group consisting of: halo, C₁-C₆ alkyl, and C₃-C₆ cycloalkyl;
   each Re is independently selected from the group consisting of: optionally substituted C₁-C₆ alkyl, optionally substituted phenyl, optionally substituted heteroaryl, optionally substituted C₅-C₇ cycloalkyl, and optionally substituted heterocycloalkyl, where said C₁-C₆ alkyl is optionally substituted with one substituent selected from the group consisting of: ORc, trifluoromethyl, phenyl, heteroaryl, heterocycloalkyl optionally substituted with ORc or heterocycloalkyl, and NRaRb; where said phenyl and heteroaryl are optionally substituted with one to three substituents each independently selected from the group consisting of: halo, CN, C₁-C₆ alkyl, C₁-C₆ haloalkyl, N(Rb)C(O)Ra, and ORf; and where said C₅-C₇ cycloalkyl and heterocycloalkyl are optionally substituted with one to three substituents each independently selected from the group consisting of: halo, C₁-C₆ alkyl optionally substituted with ORc and C₃-C₆ cycloalkyl;
   each Rf is independently selected from the group consisting of: H and C₁-C₆ alkyl;
   each Rg is independently selected from the group consisting of: H, C₁-C₆ alkyl, C₃-C₆ cycloalkyl, heteroaryl, and phenyl;
   each Rh is independently selected from the group consisting of: H and C₁-C₆ alkyl optionally substituted with one phenyl group;
   each Ri is independently selected from the group consisting of: H, C₁-C₆ alkyl, C₁-C₆ haloalkyl, and phenyl;
   Rj is optionally substituted aryl, optionally substituted heteroaryl, optionally substituted C₃-C₆ cycloalkyl, or optionally substituted heterocycloalkyl,
wherein said aryl and heteroaryl are optionally substituted with one to three substituents each independently selected from the group consisting of: —ORf, nitro, cyano, CF₃, unsubstituted C₁-C₆ alkyl, —C(O)Rf, —COORf, —NRfRg, —NHC(O)Rf, —C(O)NRfRg, —S(O)₂Rf, —NHS(O)₂Rf, and —S(O)₂NRfRg; and where said C₃-C₆ cycloalkyl and heterocycloalkyl are optionally substituted with one to three substituents each independently selected from the group consisting of: —OH, oxo, C₁-C₆ alkyl, and C₁-C₆ haloalkyl; and
   each Rx and Ry taken together with the nitrogen atom to which they are attached form a ring having from 5 to 7 member atoms wherein said ring optionally contains one additional heteroatom as a member atom, said ring is saturated or unsaturated but not aromatic, and said ring is optionally substituted with one or two C₁-C₃ alkyl substituents.

In one embodiment R1 is optionally substituted phenyl.

In one embodiment R1 is phenyl.

In one embodiment R2 is the group —YZ.

In one embodiment Y is a bond.

In one embodiment Z is a heterocycloalkyl group optionally substituted by one R3 group.

In one embodiment Z is piperidinyl optionally substituted by one R3 group.

In one embodiment Z is piperidinyl substituted by one R3 group.

In one embodiment R3 is R4, —S(O)₂R4, —C(O)R4, or —C(O)OR4.

In one embodiment R4 is optionally substituted phenyl, optionally substituted heteroaryl, or optionally substituted C₁-C₆ alkyl.

In one embodiment R4 is C₁-C₆ alkyl, phenyl, 4-fluorophenyl, or 1-methyl-1H-imidazol-4-yl.

In one embodiment R1 is phenyl; R2 is the group —YZ; Y is a bond; Z is piperidinyl substituted by one R3 group; R3 is R4, —S(O)₂R4, —C(O)R4, or —C(O)OR4; and R4 is C₁-C₆ alkyl, phenyl, 4-fluorophenyl, or 1-methyl-1H-imidazol-4-yl.

Examples of the present invention include:
1,1-dimethylethyl-4-[4-(aminocarbonyl)-6-phenyl-1H-indazol-1-yl]-1-piperidinecarboxylate;
6-phenyl-1-(4-piperidinyl)-1H-indazole-4-carboxamide;
6-phenyl-1H-indazole-4-carboxamide;
1-{1-[(4-fluorophenyl)sulfonyl]-4-piperidinyl}-6-phenyl-1H-indazole-4-carboxamide;
1-{1-[(1-methyl-1H-imidazol-4-yl)sulfonyl]-4-piperidinyl}-6-phenyl-1H-indazole-4-carboxamide;
6-phenyl-1-(1-propanoyl-4-piperidinyl)-1H-indazole-4-carboxamide;
1-[1-(ethylsulfonyl)-4-piperidinyl]-6-phenyl-1H-indazole-4-carboxamide;
6-phenyl-1-[1-(phenylcarbonyl)-4-piperidinyl]-1H-indazole-4-carboxamide;
6-phenyl-1-[1-(phenylsulfonyl)-4-piperidinyl]-1H-indazole-4-carboxamide;
6-phenyl-1-[1-(phenylmethyl)-4-piperidinyl]-1H-indazole-4-carboxamide;
1-(1-ethyl-4-piperidinyl)-6-phenyl-1H-indazole-4-carboxamide;
1,1-dimethylethyl-3-[4-(aminocarbonyl)-6-phenyl-1H-indazol-1-yl]-1-piperidinecarboxylate;
6-phenyl-1-(3-piperidinyl)-1H-indazole-4-carboxamide;
1-[1-(ethylsulfonyl)-3-piperidinyl]-6-phenyl-1H-indazole-4-carboxamide;
1-{1-[(1-methyl-1H-imidazol-4-yl)sulfonyl]-3-piperidinyl}-6-phenyl-1H-indazole-4-carboxamide;
6-phenyl-1-(1-propanoyl-3-piperidinyl)-1H-indazole-4-carboxamide;
6-phenyl-1-[1-(phenylcarbonyl)-3-piperidinyl]-1H-indazole-4-carboxamide;
6-phenyl-1-[1-(phenylsulfonyl)-3-piperidinyl]-1H-indazole-4-carboxamide; and
1-{1-[(4-fluorophenyl)sulfonyl]-3-piperidinyl}-6-phenyl-1H-indazole-4-carboxamide.

Another embodiment of the invention are compounds that are intermediates useful in the preparation of IKK-2 inhibitors. The following compounds are intermediates useful in the preparation of IKK-2 inhibitors:
1-(1-{[(1,1-dimethylethyl)oxy]carbonyl}-4-piperidinyl)-6-phenyl-1H-indazole-4-carboxylic acid;
methyl 1-(1-{[(1,1-dimethylethyl)oxy]carbonyl}-4-piperidinyl)-6-phenyl-1H-indazole-4-carboxylate;
methyl 6-phenyl-1H-indazole-4-carboxylate;
methyl 1-acetyl-6-phenyl-1H-indazole-4-carboxylate;
1-(1-{[(1,1-dimethylethyl)oxy]carbonyl}-3-piperidinyl)-6-phenyl-1H-indazole-4-carboxylic acid; and
methyl 1-(1-{[(1,1-dimethylethyl)oxy]carbonyl}-3-piperidinyl)-6-phenyl-1H-indazole-4-carboxylate.

### Terms and Definitions

“Alkyl” refers to a saturated hydrocarbon chain having the specified number of member atoms. For example, C₁-C₆ alkyl refers to an alkyl group having from 1 to 6 member atoms. Alkyl groups may be optionally substituted with one or more substituents as defined herein. Alkyl groups may be straight or branched. Representative branched alkyl groups have one, two, or three branches. Alkyl includes methyl, ethyl, propyl (n-propyl and isopropyl), butyl (n-butyl, isobutyl, and t-butyl), pentyl (n-pentyl, isopentyl, and neopentyl), and hexyl.

“Alkylene” refers to a saturated divalent hydrocarbon chain having the specified number of member atoms. For example, C₁-C₆ alkylene refers to an alkylene group having from 1 to 6 member atoms. Alkylene groups may be optionally substituted with one or more substituents as defined herein. Alkylene groups may be straight or branched. Representative branched alkylene groups have one, two, or three branches. Alkylene includes methylene, ethylene, propylene (n-propylene and isopropylene), butylene (n-butylene, isobutylene, and t-butylene), pentylene (n-pentylene, isopentylene, and neopentylene), and hexylene.

“Aryl” refers to an aromatic hydrocarbon ring. Aryl groups are monocyclic ring systems or bicyclic ring systems. Monocyclic aryl ring refers to phenyl. Bicyclic aryl rings refer to napthyl and rings wherein phenyl is fused to a cycloalkyl or cycloalkenyl ring having 5, 6, or 7 member atoms. Aryl groups may be optionally substituted with one or more substituents as defined herein.

“Cycloalkyl” refers to a saturated hydrocarbon ring having the specified number of member atoms. Cycloalkyl groups are monocyclic ring systems. For example, C₃-C₆ cycloalkyl refers to a cycloalkyl group having from 3 to 6 member atoms. Cycloalkyl groups may be optionally substituted with one or more substituents as defined herein. Cycloalkyl includes cyclopropyl, cyclobutyl, cyclopentyl, and cyclohexyl.

“Halo” refers to the halogen radical fluoro, chloro, bromo, or iodo.

“Haloalkyl” refers to an alkyl group wherein at least one hydrogen atom attached to a member atom within the alkyl group is replaced with halo. Haloalkyl includes trifluoromethyl.

“Heteroaryl” refers to an aromatic ring containing from 1 to 4 heteroatoms as member atoms in the ring. Heteroaryl groups containing more than one heteroatom may contain different heteroatoms. Heteroaryl groups may be optionally substituted with one or more substituents as defined herein. Heteroaryl groups are monocyclic ring systems or are fused, spiro, or bridged bicyclic ring systems. Monocyclic heteroaryl rings have 5 or 6 member atoms. Bicyclic heteroaryl rings have from 7 to 11 member atoms. Bicyclic heteroaryl rings include those rings wherein phenyl and a monocyclic heterocycloalkyl ring are attached forming a fused, spiro, or bridged bicyclic ring system, and those rings wherein a monocyclic heteroaryl ring and a monocyclic cycloalkyl, cycloalkenyl, heterocycloalkyl, or heteroaryl ring are attached forming a fused, spiro, or bridged bicyclic ring system. Heteroaryl includes pyrrolyl, pyrazolyl, imidazolyl, oxazolyl, isoxazolyl, thiazolyl, isothiazolyl, furanyl, furazanyl, thienyl, triazolyl, pyridinyl, pyrimidinyl, pyridazinyl, pyrazinyl, triazinyl, tetrazinyl, indolyl, isoindolyl, indolizinyl, indazolyl, purinyl, quinolinyl, isoquinolinyl, quinoxalinyl, quinazolinyl, pteridinyl, cinnolinyl, benzimidazolyl, benopyranyl, benzoxazolyl, benzofuranyl, isobenzofuranyl, benzothiazolyl, benzothienyl, furopyridinyl, and napthyridinyl.

“Heteroatom” refers to a nitrogen, sulphur, or oxygen atom.

“Heterocycloalkyl” refers to a saturated or unsaturated ring containing from 1 to 4 heteroatoms as member atoms in the ring. However, heterocycloalkyl rings are not aromatic. Heterocycloalkyl groups containing more than one heteroatom may contain different heteroatoms. Heterocycloalkyl groups may be optionally substituted with one or more substituents as defined herein. Heterocycloalkyl groups are monocyclic ring systems having from 4 to 7 member atoms or a heterocycloalkyl group can be the bicyclic ring system decahydroisoquinoline. In certain embodiments, heterocycloalkyl is saturated. In other embodiments, heterocycloalkyl is unsaturated but not aromatic. Heterocycloalkyl includes pyrrolidinyl, tetrahydrofuranyl, dihydrofuranyl, pyranyl, tetrahydropyranyl, dihydropyranyl, tetrahydrothienyl, pyrazolidinyl, oxazolidinyl, thiazolidinyl, piperidinyl, homopiperidinyl, piperazinyl, morpholinyl, thiamorpholinyl, 1,3-dioxolanyl, 1,3-dioxanyl, 1,4-dioxanyl, 1,3-oxathiolanyl, 1,3-oxathianyl, 1,3-dithianyl, and azetidinyl.

“Member atoms” refers to the atom or atoms that form a chain or ring. Where more than one member atom is present in a chain and within a ring, each member atom is covalently bound to an adjacent member atom in the chain or ring. Atoms that make up a substituent group on a chain or ring are not member atoms in the chain or ring.

“Optionally substituted” indicates that a group, such as alkyl, aryl, cycloalkyl, heterocycloalkyl, or heteroaryl, may be unsubstituted or substituted with one or more substituents as defined herein. “Substituted” in reference to a group indicates that a hydrogen atom attached to a member atom within a group is replaced. It should be understood that the term “substituted” includes the implicit provision that such substitution be in accordance with the permitted valence of the substituted atom and the substituent and that the substitution results in a stable compound (i.e. one that does not spontaneously undergo transformation such as by rearrangement, cyclization, or elimination). In certain embodiments, a single atom may be substituted with more than one substituent as long as such substitution is in accordance with the permitted valence of the atom. Suitable substituents are defined herein for each substituted or optionally substituted group.

“Pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

| | |
|---|---|
| g (grams); | mg (milligrams); |
| L (liters); | mL (milliliters); |
| μL (microliters); | psi (pounds per square |
| M (molar); | inch); |
| i.v. (intravenous); | mM (millimolar); |
| MHz (megahertz); | Hz (Hertz); |
| mmol (millimoles); | mol (moles); |
| min (minutes); | rt (room temperature); |
| mp (melting point); | h (hours); |
| Tᵣ (retention time); | TLC (thin layer chromatog- |
| MeOH (methanol); | raphy); |
| TEA (triethylamine); | RP (reverse phase); |
| TFAA (trifluoroacetic anhydride); | i-PrOH (isopropanol); |
| DMSO (dimethylsulfoxide); | TFA (trifluoroacetic acid); |
| DME (1,2-dimethoxyethane); | THF (tetrahydrofuran); |
| DCE (dichloroethane); | AcOEt (ethyl acetate); |
| DMPU (N,N′-dimethylpropyleneurea); | DCM (dichloromethane); |
| IBCF (isobutyl chloroformate); | DMF (N,N-dimethylformamide); |
| HOSu (N-hydroxysuccinimide); | CDI (1,1-carbonyldiimidazole); |
| mCPBA (meta-chloroperbenzoic acid; | HOAc (acetic acid); |
| EDC (1-[3-dimethylamino) propyl]-3- | HOBT (1-hydroxybenzo- |
| ethylcarbodiimide hydrochloride); | triazole); |
| BOC (tert-butyloxycarbonyl); | FMOC (9-fluorenylmethoxy- |
| DCC (dicyclohexylcarbodiimide); | carbonyl); |
| Ac (acetyl); | CBZ (benzyloxycarbonyl); |
| TMSE (2-(trimethylsilyl)ethyl); | atm (atmosphere); |
| TIPS (triisopropylsilyl); | TMS (trimethylsilyl); |
| DMAP (4-dimethylaminopyridine); | TBS (t-butyldimethylsilyl); |
| ATP (adenosine triphosphate); | BSA (bovine serum albumin); |
| DMEM (Dulbecco's modified Eagle | HRP (horseradish peroxidase); |
| medium); | |

HPLC (high pressure liquid chromatography);
BOP (bis(2-oxo-3-oxazolidinyl)phosphinic chloride);
TBAF (tetra-n-butylammonium fluoride);
HBTU(O-Benzotriazole-1-yl-N,N,N′,N′-tetramethyluroniumhexafluoro
phosphate);
HEPES (4-(2-hydroxyethyl)-1-piperazine ethane sulfonic acid);
DPPA (diphenylphosphoryl azide);
fHNO₃ (fuming HNO₃);
EDTA (ethylenediaminetetraacetic acid);
TMEDA (N,N,N′,N′-tetramethyl-1,2-ethanediamine);
NBS (N-bromosuccinimide);
HATU (O-(7azabenzobenzotriazol-1-yl)-N,N,N′,N′-tetramethyluronium
hexafluorophosphate);
DIPEA (diisopropylethylamine);
Imes (1,3-Bis(2,4,6-trimethylphenyl)imidazolium chloride);
dppf (1,1′-bis(diphenylphosphino)ferrocene);
CLR (Controlled Laboratory Reactor); and
NIS (N-iodsuccinimide).

All references to ether are to diethyl ether and brine refers to a saturated aqueous solution of NaCl.

The compounds according to formula I may contain one or more asymmetric center (also referred to as a chiral center) and may, therefore, exist as individual enantiomers, diastereomers, or other stereoisomeric forms, or as mixtures thereof. Chiral centers, such as chiral carbon atoms, may also be present in a substituent such as an alkyl group. Where the stereochemistry of a chiral center present in formula I, or in any chemical structure illustrated herein, is not specified the structure is intended to encompass any stereoisomer and all mixtures thereof. Thus, compounds according to formula I containing one or more chiral center may be used as racemic mixtures, enantiomerically enriched mixtures, or as enantiomerically pure individual stereoisomers.

Individual stereoisomers of a compound according to formula I which contain one or more asymmetric center may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

The compounds according to formula I may also contain double bonds or other centers of geometric asymmetry. Where the stereochemistry of a center of geometric asymmetry present in formula I, or in any chemical structure illustrated herein, is not specified, the structure is intended to encompass the trans (E) geometric isomer, the cis (Z) geometric isomer, and all mixtures thereof. Likewise, all tautomeric forms are also included in formula I whether such tautomers exist in equilibrium or predominately in one form.

The skilled artisan will appreciate that pharmaceutically-acceptable salts of the compounds according to formula I may be prepared. Indeed, in certain embodiments of the invention, pharmaceutically-acceptable salts of the compounds according to formula I may be preferred over the respective free base or free acid because such salts impart greater stability or solubility to the molecule thereby facilitating formulation into a dosage form. Accordingly, the invention is further directed to pharmaceutically-acceptable salts of the compounds according to formula I.

As used herein, the term “pharmaceutically-acceptable salts” refers to salts that retain the desired biological activity of the subject compound and exhibit minimal undesired toxicological effects. These pharmaceutically-acceptable salts may be prepared in situ during the final isolation and purification of the compound, or by separately reacting the purified compound in its free acid or free base form with a suitable base or acid, respectively.

In certain embodiments, compounds according to formula I may contain an acidic functional group. Suitable pharmaceutically-acceptable salts include salts of such acidic functional groups. Representative salts include pharmaceutically-acceptable metal salts such as sodium, potassium, lithium, calcium, magnesium, aluminum, and zinc salts; carbonates and bicarbonates of a pharmaceutically-acceptable metal cation such as sodium, potassium, lithium, calcium, magnesium, aluminum, and zinc; pharmaceutically-acceptable organic primary, secondary, and tertiary amines including aliphatic amines, aromatic amines, aliphatic diamines, and hydroxy alkylamines such as methylamine, ethylamine, 2-hydroxyethylamine, diethylamine, triethylamine, ethylenediamine, ethanolamine, diethanolamine, and cyclohexylamine.

In certain embodiments, compounds according to formula I may contain a basic functional group and are therefore capable of forming pharmaceutically-acceptable acid addition salts by treatment with a suitable acid. Suitable acids include pharmaceutically-acceptable inorganic acids and pharmaceutically-acceptable organic acids. Representative pharmaceutically-acceptable acid addition salts include hydrochloride, hydrobromide, nitrate, methylnitrate, sulfate, bisulfate, sulfamate, phosphate, acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), and napthalene-2-sulfonate.

As used herein, the term “compounds of the invention” means both the compounds according to formula I and the pharmaceutically-acceptable salts thereof.

The compounds of the invention may exist in solid or liquid form. In the solid state, the compounds of the invention may exist in crystalline or noncrystalline form, or as a mixture thereof. For compounds of the invention that are in crystalline form, the skilled artisan will appreciate that pharmaceutically-acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

The skilled artisan will further appreciate that certain compounds of the invention that exist in crystalline form, including the various solvates thereof, may exhibit polymorphism (i.e. the capacity to occur in different crystalline structures). These different crystalline forms are typically known as “polymorphs.” The invention includes all such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions.

### Compound Preparation

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the Examples section.

Compounds of formula I can be prepared, for example, according to Scheme 1 depicted below:

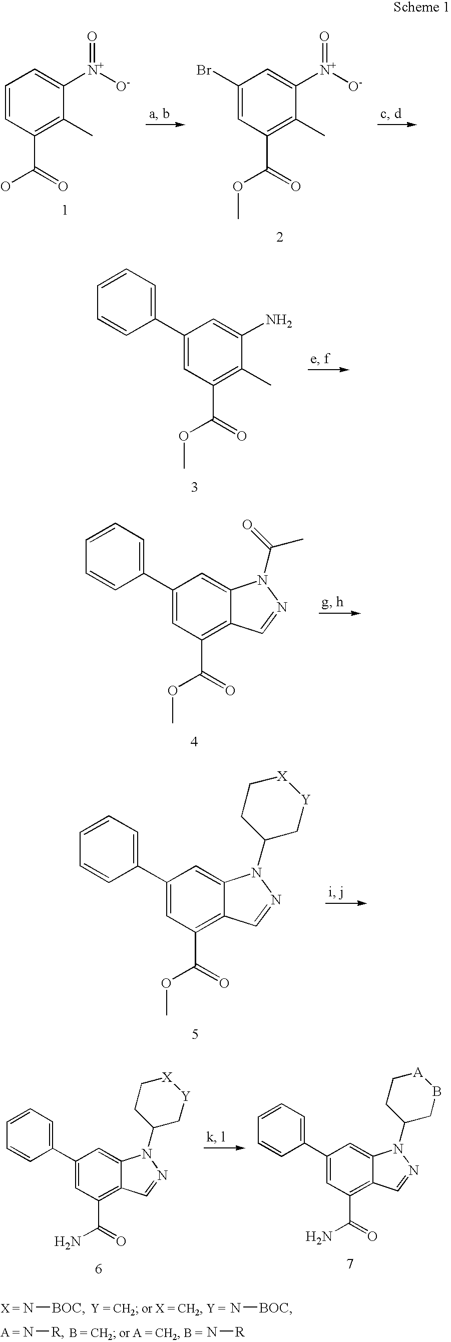

Conditions: (a) 1,3-dibromo-5,5-dimethyl-2,4-imidazolidinedione, H₂SO₄ (con.); (b) MeI, Na₂CO₃, DMF, 60° C.; (c) PhB(OH)₂, Pd(PPh₃)₄, toluene, H₂O, K₂CO₃, reflux; (d) Pd/C, HCOONH₄, EtOH, 60° C.; (e) KOAc, Ac₂O, rt; (f) 18-C-6, t-BuONO, reflux; (g) HCl, MeOH, 60° C.; (h) NaH, DMF, 1,1-dimethylethyl 4-{[(4-methylphenyl)sulfonyl]oxy}-1-piperidinecarboxylate, 95° C.; (i) NaOH, MeOH, H₂O, reflux; (j) HATU, NH₄Cl, Hunig's base, DMF;(k) HCl, MeOH; (I) RCl, DMAP, TEA or aldehyde, HOAc, NaCNBH₃

The synthesis of the N-linked indazoles began with bromonation of 2-methyl-3-nitrobenzoic acid 1 in sulfuric acid at room temperature, and ester formation with methyl iodide to provide benzoate 2. The biphenylcarboxylate 3 was obtained via Suzuki coupling with phenylboronic acid, followed by reduction with HCOONH₄ and Pd/C. With intermediate 3 in hand, acylation with acetic anhydride and cyclization using t-Butyl nitrite proceeded to give key intermediate 4. Hydrolysis of indazole 4 with HCl provided the unsubstituted indazole core, which was alkylated with piperidine sulfonate to give N-linked indazole 5. Further transformation of the ester moiety to primary carboxamide 6 was accomplished via basic hydrolysis of the methyl ester, and subsequent HTAU mediated amide formation. Deprotection of Boc group of carboxamide 6, followed by coupling reactions with appropriate electrophilic reagents provided the desired product 7.

The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999). In some instances, a substituent may be specifically selected to be reactive under the reaction conditions used. Under these circumstances, the reaction conditions convert the selected substituent into another substituent that is either useful as an intermediate compound or is a desired substituent in a target compound.

### Methods of Use

The compounds of the invention are inhibitors of IKK2. These compounds can be useful in the treatment of disorders wherein the underlying pathology is (at least in part) attributable to inappropriate IKK2 (also known as IKKβ) activity such as rheumatoid arthritis, inflammatory bowel disease, asthma, and COPD (chronic obstructive pulmonary disease). “Inappropriate IKK2 activity” refers to any IKK2 activity that deviates from the normal IKK2 activity expected in a particular patient. Inappropriate IKK2 activity may take the form of, for instance, an abnormal increase in activity, or an aberration in the timing and or control of IKK2 activity. Such inappropriate activity may result then, for example, from overexpression or mutation of the protein kinase leading to inappropriate or uncontrolled activation. Accordingly, in another aspect the invention is directed to methods of treating such disorders.

Such disorders include inflammatory and tissue repair disorders, particularly rheumatoid arthritis, inflammatory bowel disease, asthma and COPD (chronic obstructive pulmonary disease); osteoarthritis, osteoporosis and fibrotic diseases; dermatosis, including psoriasis, atopic dermatitis and ultraviolet radiation (UV)-induced skin damage; autoimmune diseases including systemic lupus eythematosus, multiple sclerosis, psoriatic arthritis, alkylosing spondylitis, tissue and organ rejection, Alzheimer's disease, stroke, atherosclerosis, restonosis, diabetes, glomerulonephritis, cancer, including Hodgkins disease, cachexia, inflammation associated with infection and certain viral infections, including acquired immune deficiency syndrome (AIDS), adult respiratory distress syndrome, and Ataxia Telangiestasia.

The methods of treatment of the invention comprise administering a safe and effective amount of a compound according to formula I or a pharmaceutically-acceptable salt thereof to a patient in need thereof. Individual embodiments of the invention include methods of treating any one of the above-mentioned disorders by administering a safe and effective amount of a compound according to formula I or a pharmaceutically-acceptable salt thereof to a patient in need thereof.

As used herein, “treat” in reference to a disorder means: (1) to ameliorate or prevent the disorder or one or more of the biological manifestations of the disorder, (2) to interfere with (a) one or more points in the biological cascade that leads to or is responsible for the disorder or (b) one or more of the biological manifestations of the disorder, (3) to alleviate one or more of the symptoms or effects associated with the disorder, or (4) to slow the progression of the disorder or one or more of the biological manifestations of the disorder.

As indicated above, “treatment” of a disorder includes prevention of the disorder. The skilled artisan will appreciate that “prevention” is not an absolute term. In medicine, “prevention” is understood to refer to the prophylactic administration of a drug to substantially diminish the likelihood or severity of a disorder or biological manifestation thereof, or to delay the onset of such disorder or biological manifestation thereof.

As used herein, “safe and effective amount” in reference to a compound of the invention or other pharmaceutically-active agent means an amount of the compound sufficient to treat the patient's condition but low enough to avoid serious side effects (at a reasonable benefit/risk ratio) within the scope of sound medical judgment. A safe and effective amount of a compound will vary with the particular compound chosen (e.g. consider the potency, efficacy, and half-life of the compound); the route of administration chosen; the disorder being treated; the severity of the disorder being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

As used herein, “patient” refers to a human or other animal.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin as well as intraocular, otic, intravaginal, and intranasal administration.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the disorder being treated, the severity of the disorder being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Typical daily dosages may vary depending upon the particular route of administration chosen. Typical daily dosages for oral administration range from 0.001 mg to 50 mg per kg of total body weight.

Additionally, the compounds of the invention may be administered as prodrugs. As used herein, a “prodrug” of a compound of the invention is a functional derivative of the compound which, upon administration to a patient, eventually liberates the compound of the invention in vivo. Administration of a compound of the invention as a prodrug may enable the skilled artisan to do one or more of the following: (a) modify the onset of the compound in vivo; (b) modify the duration of action of the compound in vivo; (C) modify the transportation or distribution of the compound in vivo; (d) modify the solubility of the compound in vivo; and (e) overcome or overcome a side effect or other difficulty encountered with the compound. Typical functional derivatives used to prepare prodrugs include modifications of the compound that are chemically or enzymatically cleaved in vivo. Such modifications, which include the preparation of phosphates, amides, esters, thioesters, carbonates, and carbamates, are well known to those skilled in the art.

The invention also provides a compound of the invention for use in medical therapy, and particularly in the treatment of disorders mediated by IKK2 activity. Thus, in a further aspect, the invention is directed to the use of a compound according to formula I or a pharmaceutically-acceptable salt thereof in the preparation of a medicament for the treatment of a disorder characterized by inappropriate IKK2 activity.

Particular disorders characterised by inappropriate IKK2 activity include inflammatory and tissue repair disorders, particularly rheumatoid arthritis, inflammatory bowel disease, asthma and COPD (chronic obstructive pulmonary disease);osteoarthritis, osteoporosis and fibrotic diseases; dermatosis, including psoriasis, atopic dermatitis and ultraviolet radiation (UV)-induced skin damage; autoimmune diseases including systemic lupus eythematosus, multiple sclerosis, psoriatic arthritis, alkylosing spondylitis, tissue and organ rejection, Alzheimer's disease, stroke, atherosclerosis, restenosis, diabetes, glomerulonephritis, cancer, including Hodgkins disease, cachexia, inflammation associated with infection and certain viral infections, including acquired immune deficiency syndrome (AIDS), adult respiratory distress syndrome, and Ataxia Telangiestasia as a result of inhibition of the protein kinase IKK2.

### Compositions

The compounds of the invention will normally, but not necessarily, be formulated into pharmaceutical compositions prior to administration to a patient. Accordingly, in another aspect the invention is directed to pharmaceutical compositions comprising a compound of the invention and one or more pharmaceutically-acceptable excipients.

The pharmaceutical compositions of the invention may be prepared and packaged in bulk form wherein a safe and effective amount of a compound of the invention can be extracted and then given to the patient such as with powders or syrups. Alternatively, the pharmaceutical compositions of the invention may be prepared and packaged in unit dosage form wherein each physically discrete unit contains a safe and effective amount of a compound of the invention. When prepared in unit dosage form, the pharmaceutical compositions of the invention typically may contain, for example, from 0.5 mg to 1 g, or from 1 mg to 700 mg, or from 5 mg to 100 mg of a compound of the invention.

The pharmaceutical compositions of the invention typically contain one compound of the invention. However, in certain embodiments, the pharmaceutical compositions of the invention contain more than one compound of the invention. For example, in certain embodiments the pharmaceutical compositions of the invention contain two compounds of the invention. In addition, the pharmaceutical compositions of the invention may optionally further comprise one or more additional pharmaceutically active compounds.

As used herein, “pharmaceutically-acceptable excipient” means a pharmaceutically acceptable material, composition or vehicle involved in giving form or consistency to the pharmaceutical composition. Each excipient must be compatible with the other ingredients of the pharmaceutical composition when comingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically-acceptable.

The compound of the invention and the pharmaceutically-acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols, solutions, and dry powders; and (6) topical administration such as creams, ointments, lotions, solutions, pastes, sprays, foams, and gels.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting the compound or compounds of the invention once administered to the patient from one organ, or portion of the body, to another organ, or portion of the body. Certain pharmaceutically-acceptable excipients may be chosen for their ability to enhance patient compliance.

Suitable pharmaceutically-acceptable excipients include the following types of excipients: Diluents, fillers, binders, disintegrants, lubricants, glidants, granulating agents, coating agents, wetting agents, solvents, co-solvents, suspending agents, emulsifiers, sweetners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a safe and effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesuim stearate, calcium stearate, and talc.

Where appropriate, dosage unit formulations for oral administration can be microencapsulated. The composition can also be prepared to prolong or sustain the release as for example by coating or embedding particulate material in polymers, wax or the like.

The compounds of the invention may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds of the invention may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

In another aspect, the invention is directed to a liquid oral dosage form. Oral liquids such as solution, syrups and elixirs can be prepared in dosage unit form so that a given quantity contains a predetermined amount of a compound of the invention. Syrups can be prepared by dissolving the compound of the invention in a suitably flavored aqueous solution, while elixirs are prepared through the use of a non-toxic alcoholic vehicle. Suspensions can be formulated by dispersing the compound of the invention in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

In another aspect, the invention is directed to a dosage form adapted for administration to a patient by inhalation. For example, the compound of the invention may be inhaled into the lungs as a dry powder, an aerosol, a suspension, or a solution.

Dry powder compositions for delivery to the lung by inhalation typically comprise a compound of the invention as a finely divided powder together with one or more pharmaceutically-acceptable excipients as finely divided powders. Pharmaceutically-acceptable excipients particularly suited for use in dry powders are known to those skilled in the art and include lactose, starch, mannitol, and mono-, di-, and polysaccharides.

The dry powder may be administered to the patient via a reservoir dry powder inhaler (RDPI) having a reservoir suitable for storing multiple (un-metered doses) of medicament in dry powder form. RDPIs typically include a means for metering each medicament dose from the reservoir to a delivery position. For example, the metering means may comprise a metering cup, which is movable from a first position where the cup may be filled with medicament from the reservoir to a second position where the metered medicament dose is made available to the patient for inhalation.

Alternatively, the dry powder may be presented in capsules (e.g. gelatin or plastic), cartridges, or blister packs for use in a multi-dose dry powder inhaler (MDPI). MDPIs are inhalers wherein the medicament is comprised within a multi-dose pack containing (or otherwise carrying) multiple defined doses (or parts thereof) of medicament. When the dry powder is presented as a blister pack, it comprises multiple blisters for containment of the medicament in dry powder form. The blisters are typically arranged in regular fashion for ease of release of the medicament therefrom. For example, the blisters may be arranged in a generally circular fashion on a disc-form blister pack, or the blisters may be elongate in form, for example comprising a strip or a tape. Each capsule, cartridge, or blister may, for example, contain between 20 μg-10 mg of the compound of the invention.

Aerosols may be formed by suspending or dissolving a compound of the invention in a liquified propellant. Suitable propellants include halocarbons, hydrocarbons, and other liquified gases. Representative propellants include: trichlorofluoromethane (propellant 11), dichlorofluoromethane (propellant 12), dichlorotetrafluoroethane (propellant 114), tetrafluoroethane (HFA-134a), 1,1-difluoroethane (HFA-152a), difluoromethane (HFA-32), pentafluoroethane (HFA-12), heptafluoropropane (HFA-227a), perfluoropropane, perfluorobutane, perfluoropentane, butane, isobutane, and pentane. Aerosols comprising a compound of the invention will typically be administered to a patient via a metered dose inhaler (MDI). Such devices are known to those skilled in the art.

The aerosol may contain additional pharmaceutically-acceptable excipients typically used with MDls such as surfactants, lubricants, cosolvents and other excipients to improve the physical stability of the formulation, to improve valve performance, to improve solubility, or to improve taste.

Suspensions and solutions comprising a compound of the invention may also be administered to a patient via a nebulizer. The solvent or suspension agent utilized for nebulization may be any pharmaceutically-acceptable liquid such as water, aqueous saline, alcohols or glycols, e.g., ethanol, isopropylalcohol, glycerol, propylene glycol, polyethylene glycol, etc. or mixtures thereof. Saline solutions utilize salts which display little or no pharmacological activity after administration. Both organic salts, such as alkali metal or ammonium halogen salts, e.g., sodium chloride, potassium chloride or organic salts, such as potassium, sodium and ammonium salts or organic acids, e.g., ascorbic acid, citric acid, acetic acid, tartaric acid, etc. may be used for this purpose.

Other pharmaceutically-acceptable excipients may be added to the suspension or solution. The compound of the invention may be stabilized by the addition of an inorganic acid, e.g., hydrochloric acid, nitric acid, sulphuric acid and/or phosphoric acid; an organic acid, e.g., ascorbic acid, citric acid, acetic acid, and tartaric acid, etc., a complexing agent such as EDTA or citric acid and salts thereof; or an antioxidant such as antioxidant such as vitamin E or ascorbic acid. These may be used alone or together to stabilize the compound of the invention. Preservatives may be added such as benzalkonium chloride or benzoic acid and salts thereof. Surfactant may be added particularly to improve the physical stability of suspensions. These include lecithin, disodium dioctylsulphosuccinate, oleic acid and sorbitan esters.

Pharmaceutical compositions adapted for transdermal administration may be presented as discrete patches intended to remain in intimate contact with the epidermis of the patient for a prolonged period of time. For example, the active ingredient may be delivered from the patch by iontophoresis as generally described in Pharmaceutical Research, 3(6), 318 (1986).

Pharmaceutical compositions adapted for topical administration may be formulated as ointments, creams, suspensions, lotions, powders, solutions, pastes, gels, sprays, aerosols or oils.

For treatments of the eye or other external tissues, for example mouth and skin, the compositions may be applied as a topical ointment or cream. When formulated in an ointment, the compound of the invention may be employed with either a paraffinic or a water-miscible ointment base. Alternatively, the compound of the invention may be formulated in a cream with an oil-in-water cream base or a water-in-oil base.

Pharmaceutical compositions adapted for nasal administration wherein the carrier is a solid include a coarse powder having a particle size for example in the range 20 to 500 microns which is administered by rapid inhalation through the nasal passage from a container of the powder held close up to the nose. Suitable compositions wherein the carrier is a liquid, for administration as a nasal spray or as nasal drops, include aqueous or oil solutions of the compound of the invention.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

### PREPARATIONS AND EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

Unless otherwise noted, all starting materials were obtained from commercial suppliers and used without further purification. Unless otherwise indicated, all temperatures are expressed in ° C. (degrees Centigrade). All reactions are conducted under an inert atmosphere at room temperature unless otherwise noted.

¹H NMR spectra were recorded on a Brucker DPX400, a Brucker DPX250, a Brucker AC400, or a Varian Inova 400. Chemical shifts are expressed in parts per million (ppm, δ units). Splitting patterns describe apparent multiplicities and are designated as s (singlet), d (doublet), t (triplet), q (quartet), quint (quintet), m (multiplet), br (broad).

Low-resolution mass spectra (MS) were recorded on a JOEL JMS-AX505HA, JOEL SX-102, or a SCIEX-APIiii spectrometer; LC-MS were recorded on Waters ZQ or PE Sciex Single Quadrupole LC/MS API-150 spectrometers.

Preparative HPLC refers to methods where the material was purified by high performance liquid chromatography on a HPLC ABZ+5 μm column (10 cm×21.2 mm i.d.) with 0.1% formic acid in water and 0.05% formic acid in acetonitrile utilising gradient elution at a flow rate of 8 ml/min and UV detection at 254 nM.

Unless otherwise stated, silica flash column chromatography and Combiflash refers to the purification of material using Redisep™ pre-packed silica flash columns on an ISCO sq16× machine with the stated solvent systems.

Reverse phase HPLC method A refers to methods where the materials were purified by high performance liquid chromatography on an HPLC S-5 μm column (75×30 mm i.d.) utilizing gradient elution with the stated solvent systems and UV detection at 254 nm.

Reverse phase HPLC method B refers to methods where the materials was purified by high performance liquid chromatography on a HPLC Luna C18 (2) 100A column (50×21.2 mm i.d.) utilizing gradient elution with the stated solvent system and UV detection at 254 nm.

Mass spectra were recorded on the following equipment: (1) Platform LCT with electrospray source operating in positive ion mode. Waters 1525 Ic pump running at 2.0 ml/min, HTS PAL autosampler, 200 ul/min split to the ESI source with inline Waters UV2488 Dual Wavelength UV detector at 254 nm and Sedex ELS detection. Column—Higgins Clipeus C18 5 um 100×3.0 mm, or (2) Finnigan TSQ700 with electrospray source operating in positive or negative ion mode. HP1050 system running at 2.0 mL/min, 200 uL/min split to the ESI source with inline HP1050 Single Wavelength UV detector at 254 nm. Column—Higgins Clipeus C18, 5 micron, 100×3.0 mm

### (1) Preparation of 1-(1-{[(1,1-dimethylethyl)oxy]carbonyl}-4-piperidinyl)-6-phenyl-1H-indazole-4-carboxylic acid

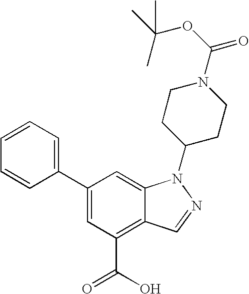

To a solution of methyl 1-(1-{[(1,1-dimethylethyl)oxy]carbonyl}-4-piperidinyl)-6-phenyl-1H-indazole-4-carboxylate (3.17 g, 7.28 mmol) in methanol (90 mL) was added 1N sodium hydroxide in water (30 mL, 30 mmol). The reaction mixture was stirred at room temperature for 5 mins and refluxed for 2 hrs. The reaction mixture was concentrated under reduced pressure. The residue was dissolved in water (250 mL) and cooled in ice bath while adding concentrate HCl dropwise until the pH value is 1. There is solid precipitated off. The solid was filtered and dried under vacuum. The solid was used in the next step without further purification.

LC/MS (ESI) 422 [M+H]⁺ Rt 3.81 min

### (2) Preparation of methyl 1-(1-{[(1,1-dimethylethyl)oxy]carbonyl}-4-piperidinyl)-6-phenyl-1H-indazole-4-carboxylate

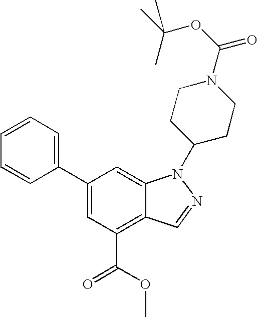

To a solution of sodium hydride (0.83 g, 20.8 mmol) in dimethylformamide (75 mL) at room temperature was added methyl 6-phenyl-1H-indazole-4-carboxylate (5 g, 19.8 mmol) in dimethylformamide (75 mL). The reaction mixture was heated to 95° C. for 2 hrs. 1,1-Dimethylethyl 4-{[(4-methylphenyl)sulfonyl]oxy}-1-piperidinecarboxylate (7.75 g, 21.8 mmol) in dimethylformamide (50 mL) was added in one portion to the reaction mixture, which was continued to heat at 95° C. overnight. The reaction mixture was cooled to room temperature and concentrated under reduced pressure. Water (600 mL) and ethyl acetate (400 mL) were added to the residue. The aqueous phase was extracted with ethyl acetate (2×400 mL).The combined organic phases were separated, washed with brine (500 mL), dried over MgSO₄, filtered and concentrated. The residue was purified by using a MPLC eluting with 3:2 n-heptane/t-butyl methyl ether to give a white solid (3.17 g, 37%)

¹H NMR (400 MHz, DMSO) δ ppm 1.45 (s, 9H) 1.97 (m, 4H) 3.05 (bs, 2H) 3.97 (s, 3H) 4.17 (d, 2H) 5.17 (m, 1H) 7.42 (m, 1H) 7.58 (m, 3H) 7.87 (d, 2H) 8.07 (s, 1H) 8.41 (s, 1H) 8.47 (s, 1H)

### (3) Preparation of methyl 6-phenyl-1H-indazole-4-carboxylate

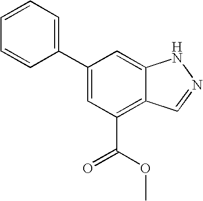

To a solution of methyl 1-acetyl-6-phenyl-1H-indazole-4-carboxylate (12.6 g, 42.8 mmol) in methanol (250 mL) was added 6N HCl in water (250 mL). The reaction mixture was heated at 60° C. for 8 hrs and concentrated under reduced pressure. The residue was adjusted pH value to 8 with concentrated ammonia. The aqueous phase was extracted with ethyl acetate (3×200 mL).The combined organic phases were separated, dried over MgSO₄ filtered and concentrated to give methyl 6-phenyl-1H-indazole-4-carboxylate (10.3 g, 95%).

LC/MS (ESI) 252.2 [M+H]⁺ Rt 3.31 min

### (4) Preparation of methyl 1-acetyl-6-phenyl-1H-indazole-4-carboxylate

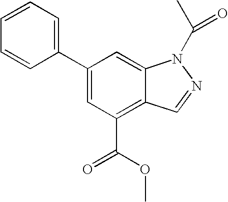

To a solution of methyl 5-amino-4-methyl-3-biphenylcarboxylate(11.8 g, 48.9 mmol) in chloroform (100 mL) was added potassium acetate (5.03 g, 51.3 mmol) and acetic anhydride (10.85 mL, 97.8 mmol). The reaction mixture was stirred at room temperature overnight. 18-Crown-6 (2.58 g, 9.78 mmol) and t-butylnitrite (12.85 mL, 108 mmol) were added to the reaction mixture, which was later refluxed overnight. The reaction mixture was cooled to room temperature, diluted with chloroform (300 mL) and washed with saturated aqueous NaHCO₃ (3×100 mL). The organic phase was dried over MgSO₄, filtered and concentrated to give methyl 1-acetyl-6-phenyl-1H-indazole-4-carboxylate (12.61 g, 88%), which was used in the next step without further purification.

LC/MS (ESI) 294[M+H]⁺ Rt 3.93 min

### (5) Preparation of methyl 5-amino-4-methyl-3-biphenylcarboxylate

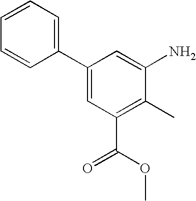

To a solution of methyl 4-methyl-5-nitro-3-biphenylcarboxylate (21.81 g, 80.4 mmol) in ethanol (600 mL) at 60-70° C. was added ammonium formate (30.42 g, 482.4 mmol) and palladium on carbon (10%) (600 mg). The reaction mixture was refluxed overnight, filtered and concentrated under reduced pressure. The residue was dissolved in water (300 mL) and ethyl acetate (500 mL). The aqueous phase was extracted with ethyl acetate (2×400 mL).The combined organic phases were separated, dried over MgSO₄ filtered and concentrated to give methyl 5-amino-4-methyl-3-biphenylcarboxylate (19.00 g, 98%), which was used in the next step without further purification.

¹H NMR (400 MHz, CDCl₃) δ ppm 2.35 (s, 3H) 3.37 (s, 3H) 7.05 (s, 1H) 7.34 (m, 1H) 7.41 (m, 2H) 7.47 (d, 1H) 7.58 (m, 2H).

### (6) Preparation of methyl 4-methyl-5-nitro-3-biphenylcarboxylate

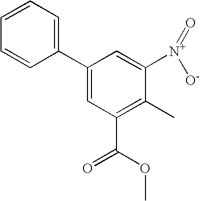

To a solution of methyl 5-bromo-2-methyl-3-nitrobenzoate (24.23 g, 93.2 mmol) in toluene (250 mL) was added tetrakis(triphenylphosphine) palladium (0) (3.23 g, 2.796 mmol) and 2N sodium carbonate solution (93 mL, 186.4 mmol). The reaction mixture was stirred vigorously at room temperature while phenylbronic acid (15.91 g, 130.5 mmol) in methanol (65 mL) was added. Then the reaction mixture was refluxed for 8 hrs, cooled to room temperature, filtered. The filtrate was diluted with water (200 mL). The aqueous phase was separated and extracted with ethyl acetate (2×300 mL).The combined organic phases were washed with 1N sodium hydroxide solution (250 mL), separated, dried over MgSO₄, filtered and concentrated to give methyl 4-methyl-5-nitro-3-biphenylcarboxylate (21.81 g, 86%), which was used in the next step without further purification.

¹H NMR (400 MHz, CDCl₃) δ ppm 2.62 (s, 3H) 3.97 (s, 3H) 7.44 (m, 3H) 7.59 (m, 2H) 8.03 (s, 1H) 8.11 (s, 1H)

### (7) Preparation of methyl 5-bromo-2-methyl-3-nitrobenzoate

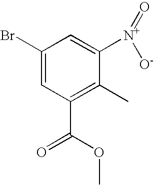

To a mixture of methyl 5-bromo-2-methyl-3-nitrobenzoic acid (5 g, 19.2 mmol) and iodomethane (4.79 mL and 76.9 mmol) in DMF (70 mL) was added sodium carbonate (6.46 g, 76.9 mmol). The reaction mixture was stirred at 60° C. for 8 hrs and then diluted with water (25 mL). The solution was filtered. The filtrate was separated and the inorganic phase was washed with ethyl acetate (3×50 mL). The combined organic phases were concentrated under reduced pressure. The resulting residue was redissoved in ethyl acetate (600 mL), washed with 5% sodium bicarbonate solution (250 mL) and 5M HCl (100 mL). The organic combined phases were washed with brine (200 mL), separated, dried over MgSO₄, filtered and concentrated to give methyl 5-bromo-2-methyl-3-nitrobenzoate (4.78 g, 92%), which was used in the next step without further purification.

LC/MS (ESI) 274 [M+H]⁺ Rt 3.76 min

### (8) Preparation of 5-bromo-2-methyl-3-nitrobenzoic acid

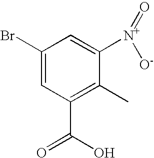

To a mixture of 2-methyl-3-nitrobenzoic acid (150 g, 820 mmol) in concentrate sulphuric acid (600 mL) was added 1,3-dibromo-5,5-dimethyl-2,4-imidazolidinedione (130.7 g, 457 mmol). The reaction mixture was stirred at room temperature for 5 hrs and then the reaction mixture was carefully added to 4 L of ice-water. The solid precipitated out, which was washed with 1.5 L water. The solid was filtered off and dried in a heated desiccator at 30-40° C. overnight to give 5-bromo-2-methyl-3-nitrobenzoic acid (220 g, 99%), which was used in the next step without further purification.

LC/MS (ESI) 257 [M+H]⁺ Rt 3.07 min

### (9) Preparation of 1,1-dimethylethyl 4-{[(4-methylphenyl)sulfonyl]oxy}-1-piperidinecarboxylate

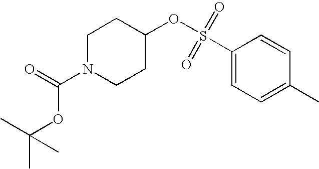

To a solution of 1,1-dimethylethyl 4-hydroxy-1-piperidinecarboxylate (5 g, 24.8 mmol) in dichloromethane (80 mL) was added p-toluenesulphonyl chloride (5.67 g, 190.65 mmol) and triethyl amine (5.15 mL, 37.2 mmol). The reaction mixture was stirred at room temperature overnight. The reaction mixture was diluted with water (50 mL) and dichloromethane (80 mL). The aqueous phase was separated and extracted with dichloromethane (2×80 mL). Pyridine was added to the combined organic phases and stood overnight. The solution was washed with 1N HCl (100 mL), separated, dried over MgSO₄ filtered and concentrated to give 1,1-dimethylethyl 4-{[(4-methylphenyl)sulfonyl]oxy}-1-piperidinecarboxylate (7.9 g, 89%), which was used in the next step without further purification.

1H NMR (400 MHz, CDCl₃) δ ppm 1.41 (s, 9H) 1.72 (m, 2H) 1.76 (m, 2H) 2.43 (s, 3H) 3.21 (m, 2H) 3.59 (m, 2H) 4.66 (m, 1H) 7.31 (d, 2H) 7.80 (d, 2H).

### (10) Preparation of 1-(1-{[(1,1-dimethylethyl)oxy]carbonyl}-3-piperidinyl)-6-phenyl-1H-indazole-4-carboxylic acid

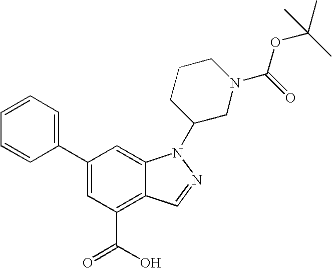

Following the same procedure of example 1, methyl 1-(1-{[(1,1-dimethylethyl) oxy]carbonyl}-3-piperidinyl)-6-phenyl-1H-indazole-4-carboxylate (intermediate 11) (1.19 g, 2.73 mmol) and 1 N sodium hydroxide in water (15 mL, 15 mmol) were reacted to give the title compound (1.06 g, 92%).

¹H NMR (400 MHz, DMSO) δ ppm 1.45 (s, 9H) 1.96 (m, 4H) 3.05 (br, 2H) 4.17 (d, 2H) 5.11 (bs, 1H) 7.45 (m, 1H) 7.58 (m, 2H) 7.80 (d, 2H) 8.06 (s, 1H) 8.37 (s, 1H) 8.43 (s, 1H) 13.31 (bs, 1H).

### (11) Preparation of methyl 1-(1-{[(1,1-dimethylethyl)oxy]carbonyl}-3-piperidinyl)-6-phenyl-1H-indazole-4-carboxylate

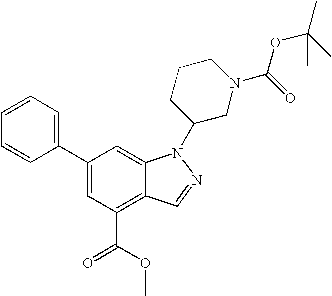

Following the same procedure of example 2, methyl 6-phenyl-1H-indazole-4-carboxylate methyl (5.00 g, 19.8 mmol), sodium hydride (0.83 g, 20.8 mmol) and 1,1-Dimethylethyl 4-{[(4-methylphenyl)sulfonyl]oxy}-1-piperidinecarboxylate (1.19 g, 21.8 mmol) were reacted to give the title compound (1.06 g, 13%).

1H NMR (400 MHz, DMSO) δ ppm 1.38 (bs, 9H) 1.65 (bs, 1H) 1.96 (bs, 1H) 2.12 (bs, 2H) 2.98 (t, 1H) 3.45 (bs, 1H) 3.97 (bs, 1H) 4.02 (s, 3H) 4.11 (bs, 1H) 4.97 (m, 1H) 7.42 (m, 1H) 7.58 (m, 2H) 7.80 (m, 2H) 8.13 (s, 1H) 8.42 (d, 2H).

### EXAMPLES

### (1) 1,1-dimethylethyl-4-[4-(aminocarbonyl)-6-phenyl-1H-indazol-1-yl]-1-piperidinecarboxylate

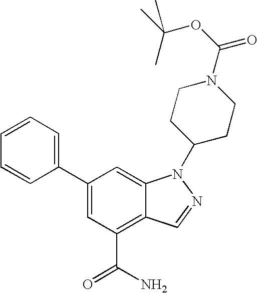

To a mixture of 1-(1-{[(1,1-dimethylethyl)oxy]carbonyl}-4-piperidinyl)-6-phenyl-1H-indazole-4-carboxylic acid (100 mg, 0.237 mmol), HATU (135 mg, 0.356 mmol) in 4 mL dimethylformamide, ammonium chloride (50 mg, 0.95 mmol) and diisopropyltheylamine (83 uL, 0.475 mmol) were added. The reaction mixture was stirred at room temperature for 1 hr and concentrated under reduced pressure. Ethyl acetate (50 mL) and water (15 mL) were added. The organic phase was separated, washed with aqueous 5% NaHCO₃ (3×10 mL) and brine (15 mL), dried over MgSO₄, filtered and concentrated. The residue was purified by using a Gilson semi-preparative HPLC system with a YMC ODS-A (C-18) column 50 mm by 20 mm ID, eluting with 40% B to 80% B in 3.2 min, hold for 1 min where A=H₂O (0.1% trifluoroacetic acid) and B=CH₃CN (0.1% trifluoroacetic acid) pumped at 25 mL/min to afford 1,1-dimethylethyl-4-[4-(aminocarbonyl)-6-phenyl-1H-indazol-1-yl]-1-piperidinecarboxylate (15 mg, 15%).

LC/MS (ESI) 420.00 [M+H]⁺ Rt 11.26 min

### (2) 6-phenyl-1-(4-piperidinyl)-1H-indazole-4-carboxamide

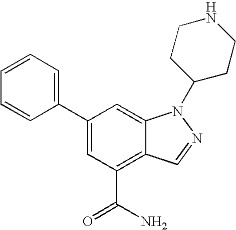

To a solution of 1,1-dimethylethyl-4-[4-(aminocarbonyl)-6-phenyl-1H-indazol-1-yl]-1-piperidinecarboxylate (6.885 mmol) in 100 mL methanol was added concentrated hydrochloric acid (5 mL). The reaction mixture was stirred at room temperature overnight and concentrated under reduced pressure. The residue was recrystallize with ethanol (20 mL) to give the title compound (1.5 g, 61%)

LC/MS (ESI) 320.00 [M+H]⁺ Rt 5.98 min

### (3) 6-phenyl-1H-indazole-4-carboxamide

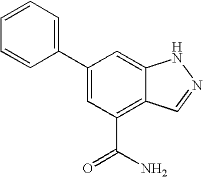

To a solution of methyl 6-phenyl-1H-indazole-4-carboxylate (100 mg, 0.397 mmol) was added concentrated ammonia (2.5 mL). The reaction mixture was heated in microwave at 120° C. for 10 mins and concentrated under reduced pressure. The residue was redissolved in dichloromethane. Oxalyl chloride (4 mL) and one drop of DMF were added. The reaction mixture was stirred at room temperature overnight. After the solution was concentrated again under reduced pressure, concentrated ammonia (3 mL) was added at room temperature. Ethyl acetate (50 mL) and water (15 mL) were added. The organic phase was separated, washed with brine (15 mL), dried over MgSO₄ filtered and concentrated. The residue was purified by using a Gilson semi-preparative HPLC system with a YMC ODS-A (C-18) column 50 mm by 20 mm ID, eluting with 40% B to 80% B in 3.2 min, hold for 1 min where A=H₂O (0.1% trifluoroacetic acid) and B=CH₃CN (0.1% trifluoroacetic acid) pumped at 25 mL/min to afford the title compound (23 mg, 24%)

LC/MS (ESI) 237.00 [M+H]⁺ Rt 7.38 min

### (4) 1-{1-[(4-fluorophenyl)sulfonyl]-4-piperidinyl}-6-phenyl-1H-indazole-4-carboxamide

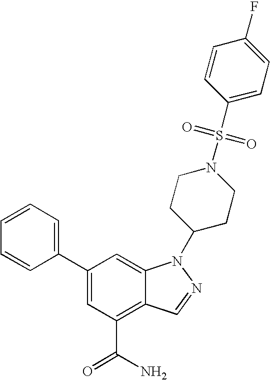

To a solution of 6-phenyl-1-(4-piperidinyl)-1H-indazole-4-carboxamide (example 2) (100 mg, 0.28 mmol) in DMF (4 mL) was added triethylamine (156 uL, 1.12 mmol), DMAP (4 mg, 0.028 mmol) and 4-fluorobenzenesulphonyl chloride (54 mg, 0.28 mmol). The reaction mixture was stirred at room temperature for 4 hrs. The solution was concentrated under reduced pressure. Ethyl acetate (50 mL) and water (15 mL) were added. The organic phase was separated, washed with brine (15 mL), dried over MgSO₄, filtered and concentrated. The residue was purified by using a Gilson semi-preparative HPLC system with a YMC ODS-A (C-18) column 50 mm by 20 mm ID, eluting with 40% B to 80% B in 3.2 min, hold for 1 min where A=H₂O (0.1% trifluoroacetic acid) and B=CH₃CN (0.1% trifluoroacetic acid) pumped at 25 mL/min to afford the title compound (26.5 mg, 20%)

LC/MS (ESI) 478.00 [M+H]⁺ Rt 11.57 min

### (5) 1-{1-[(1-methyl-1H-imidazol-4-yl)sulfonyl]-4-piperidinyl}-6-phenyl-1H-indazole-4-carboxamide

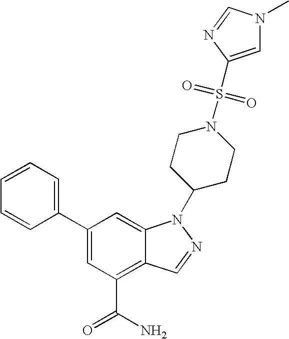

Following the general procedure of example 4,6-phenyl-1-(4-piperidinyl)-1H-indazole-4-carboxamide (example 2) (100 mg, 0.28 mmol), triethylamine (156 uL, 1.12 mmol), DMAP (4 mg, 0.028 mmol) and 1-methyl-1H-imidazole-4-sulfonyl chloride (51 mg, 0.28 mmol) were reacted to give the title compound (62.5 mg, 48%).

LC/MS: 464.00 Rt: 9.13 min.

### (6) 6-phenyl-1-(1-propanoyl-4-piperidinyl)-1H-indazole-4-carboxamide

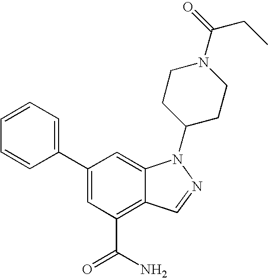

Following the general procedure of example 4,6-phenyl-1-(4-piperidinyl)-1H-indazole-4-carboxamide (example 2)(50 mg, 0.14 mmol), triethylamine (78 uL, 0.56 mmol), DMAP (4 mg, 0.028 mmol) and propanoyl chloride (12 uL, 0.14 mmol) were reacted to give the title compound (22 mg, 42%).

LC/MS: 376.00 Rt. 9.27 min.

### (7) 1-[1-(ethylsulfonyl)-4-piperidinyl]-6-phenyl-1H-indazole-4-carboxamide

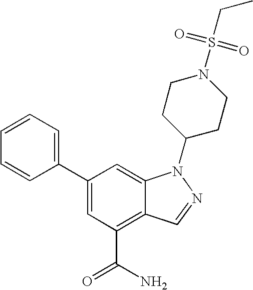

Following the general procedure of example 4,6-phenyl-1-(4-piperidinyl)-1H-indazole-4-carboxamide (example 2)(100 mg, 0.28 mmol), triethylamine (156 uL, 1.12 mmol), DMAP (4 mg, 0.028 mmol) and ethanesulphonyl chloride (26 uL, 0.28 mmol) were reacted to give the title compound (38 mg, 33%).

LC/MS: 412.00 Rt: 9.90 min.

### (8) 6-phenyl-1-[1-(phenylcarbonyl)-4-piperidinyl]-1H-indazole-4-carboxamide

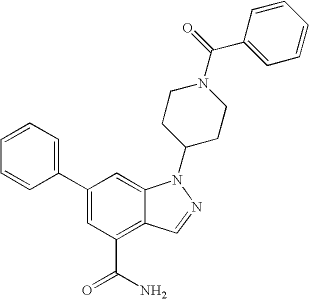

Following the general procedure of example 4,6-phenyl-1-(4-piperidinyl)-1H-indazole-4-carboxamide (example 2)(50 mg, 0.14 mmol), triethylamine (78 uL, 0.56 mmol), DMAP (2 mg, 0.014 mmol) and benzoyl chloride (16 uL, 0.14 mmol) were reacted to give the title compound (42.8 mg, 93%).

LC/MS: 424.00 Rt: 10.22 min.

### (9) 6-phenyl-1-[1-(phenylsulfonyl)-4-piperidinyl]-1H-indazole-4-carboxamide

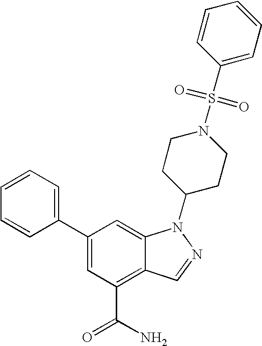

Following the general procedure of example 4,6-phenyl-1-(4-piperidinyl)-1H-indazole-4-carboxamide (example 2) (100 mg, 0.28 mmol), triethylamine (156 uL, 1.12 mmol), DMAP (4 mg, 0.028 mmol) and benzenesulphonyl chloride (36 uL, 0.28 mmol) were reacted to give the title compound (27.7 mg, 21%).

LC/MS: 460.00 Rt: 11.34 min.

### (10) 6-phenyl-1-[1-(phenylmethyl)-4-piperidinyl]-1H-indazole-4-carboxamide

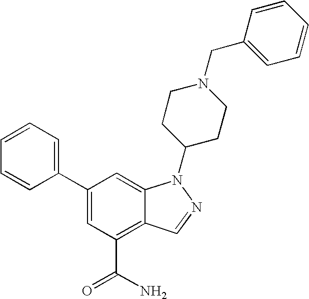

To a solution of 6-phenyl-1-(4-piperidinyl)-1H-indazole-4-carboxamide (example 2) (50 mg, 0.14 mmol) in DMF/THF (2.5 mL/0.5 mL) was added benzaldehyde (120 uL, 0.70 mmol), acetic acid (39 uL, 0.66 mmol) and MP-cyanoborohydride (2.36 mmol/g) (140 mg, 0.33 mmol). The reaction mixture was stirred at room temperature overnight. The solution was filtered and the filtrate was concentrated under reduced pressure. The residue was purified by using a Gilson semi-preparative HPLC system with a YMC ODS-A (C-18) column 50 mm by 20 mm ID, eluting with 40% B to 80% B in 3.2 min, hold for 1 min where A=H₂O (0.1% trifluoroacetic acid) and B=CH₃CN (0.1% trifluoroacetic acid) pumped at 25 mL/min to afford the title compound (7.8 mg, 14%)

LC/MS (ESI) 410.00 [M+H]⁺ Rt 6.93 min

### (11) 1-(1-ethyl-4-piperidinyl)-6-phenyl-1H-indazole-4-carboxamide

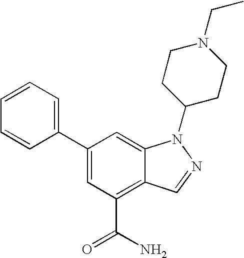

To a solution of 6-phenyl-1-(4-piperidinyl)-1H-indazole-4-carboxamide (example 2) (100 mg, 0.28 mmol) in DMF (6 mL) was added acetaldehyde (15 mg, 0.337 mmol) and MP-cyanoborohydride (2.36 mmol/g) (240 mg, 0.66 mmol). The reaction mixture was stirred at room temperature overnight. The solution was filtered and the filtrate was concentrated under reduced pressure. The residue was purified by using a Gilson semi-preparative HPLC system with a YMC ODS-A (C-18) column 50 mm by 20 mm ID, eluting with 40% B to 80% B in 3.2 min, hold for 1 min where A=H₂O (0.1% trifluoroacetic acid) and B=CH₃CN (0.1% trifluoroacetic acid) pumped at 25 mL/min to afford the title compound (31.3 mg, 32%)

LC/MS (ESI) 348.00 [M+H]⁺ Rt 6.40 min

### (12) 1,1-dimethylethyl-3-[4-(aminocarbonyl)-6-phenyl-1H-indazol-1-yl]-1-piperidinecarboxylate

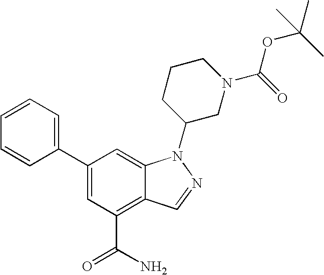

To a mixture of 1-(1-{[(1,1-dimethylethyl)oxy]carbonyl}-3-piperidinyl)-6-phenyl-1H-indazole-4-carboxylic acid (1.06 g, 2.52 mmol), HATU (1.43 g, 3.77 mmol) in 20 mL dimethylformamide, ammonium chloride (538 mg, 10.06 mmol) and diisopropyltheylamine (861 uL, 5.03 mmol) were added. The reaction mixture was stirred at room temperature overnight and concentrated under reduced pressure. Ethyl acetate (100 mL) and water (30 mL) were added. The organic phase was separated, washed with aqueous 1 N HCl (30 mL), aqueous saturated Na₂CO₃ (30 mL) and brine (50 mL), dried over MgSO₄, filtered and concentrated. The residue was purified by using a Gilson semi-preparative HPLC system with a YMC ODS-A (C-18) column 50 mm by 20 mm ID, eluting with 40% B to 80% B in 3.2 min, hold for 1 min where A=H₂O (0.1% trifluoroacetic acid) and B=CH₃CN (0.1% trifluoroacetic acid) pumped at 25 mL/min to afford the title compound (1.16 g, 99%).

LC/MS (ESI) 420.00 [M+H]⁺ Rt 11.62 min

### (13) 6-phenyl-1-(3-piperidinyl)-1H-indazole-4-carboxamide

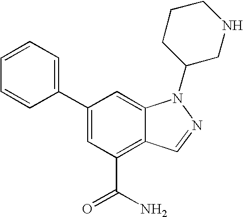

The solution of 1,1-dimethylethyl-3-[4-(aminocarbonyl)-6-phenyl-1H-indazol-1-yl]-1-piperidinecarboxylate (40 mg, 95.2 mmol) in 10 mL TFA/DCM was stirred at room temperature for 2 hrs and concentrated under reduced pressure. The residue was recrystallize with diethyl ether (20 mL) to give the title compound (34.8 mg, 99%)

LC/MS (ESI) 320.16 [M+H]⁺ Rt 6.31 min

### (14) 1-[1-(ethylsulfonyl)-3-piperidinyl]-6-phenyl-1H-indazole-4-carboxamide

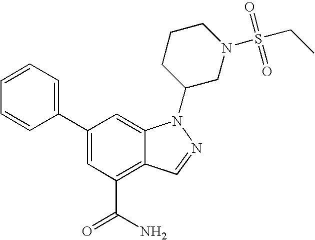

Following the general procedure of example 4,6-phenyl-1-(3-piperidinyl)-1H-indazole-4-carboxamide (example 13) (66 mg, 0.185 mmol), triethylamine (103 uL, 0.74 mmol), DMAP (4 mg, 0.028 mmol) and ethanesulphonyl chloride (17 uL, 0.185 mmol) were reacted to give the title compound (13.7 mg, 18%).

LC/MS: 412.00 Rt: 9.42 min.

### (15) 1-{1-[(1-methyl-1H-imidazol-4-yl)sulfonyl]-3-piperidinyl}-6-phenyl-1H-indazole-4-carboxamide

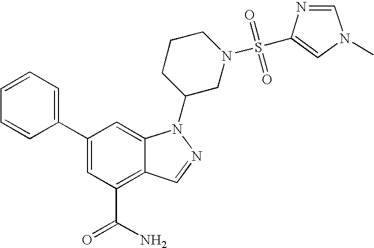

Following the general procedure of example 4, 6-phenyl-1-(3-piperidinyl)-1H-indazole-4-carboxamide (example 13) (63 mg, 0.177 mmol), triethylamine (98 uL, 0.71 mmol), DMAP (4 mg, 0.028 mmol) and 1-methyl-1H-imidazole-4-sulfonyl chloride (32 mg, 0.177 mmol) were reacted to give the title compound (18.0 mg, 22%).

LC/MS: 464.00 Rt: 8.82 min.

### (16) 6-phenyl-1-(1-propanoyl-3-piperidinyl)-1H-indazole-4-carboxamide

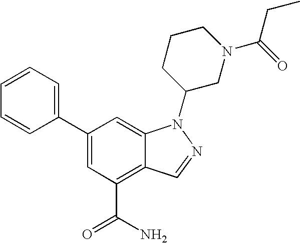

Following the general procedure of example 4, 6-phenyl-1-(3-piperidinyl)-1H-indazole-4-carboxamide (example 13) (96 mg, 0.27 mmol), triethylamine (156 uL, 1.12 mmol), DMAP (3.4 mg, 0.027 mmol) and propanoyl chloride (23 uL, 0.27 mmol) were reacted to give the title compound (26 mg, 26%).

LC/MS: 376.00 Rt: 8.94 min.

### (17) 6-phenyl-1-[1-(phenylcarbonyl)-3-piperidinyl]-1H-indazole-4-carboxamide

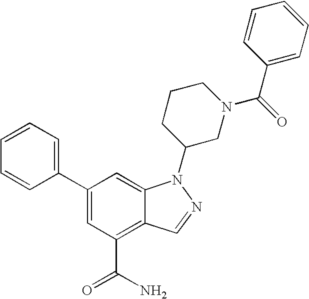

Following the general procedure of example 4, 6-phenyl-1-(3-piperidinyl)-1H-indazole-4-carboxamide (example 13) (92.5 mg, 0.26 mmol), triethylamine (156 uL, 1.12 mmol), DMAP (3.4 mg, 0.027 mmol) and benzoyl chloride (36.5 mg, 0.26 mmol) were reacted to give the title compound (31 mg, 28%).

LC/MS: 424.00 Rt: 9.77 min.

### (18) 6-phenyl-1-[1-(phenylsulfonyl)-3-piperidinyl]-1H-indazole-4-carboxamide

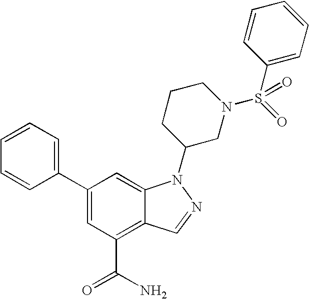

Following the general procedure of example 4, 6-phenyl-1-(3-piperidinyl)-1H-indazole-4-carboxamide (example 13) (63 mg, 0.177 mmol), triethylamine (98 uL, 0.71 mmol), DMAP (3.4 mg, 0.027 mmol) and benzenesulphonyl chloride (23 uL, 0.177 mmol) were reacted to give the title compound (21.6 mg, 27%).

LC/MS: 460.00 Rt: 10.97 min.

### (19) 1-{1-[(4-fluorophenyl)sulfonyl]-3-piperidinyl}-6-phenyl-1H-indazole-4-carboxamide

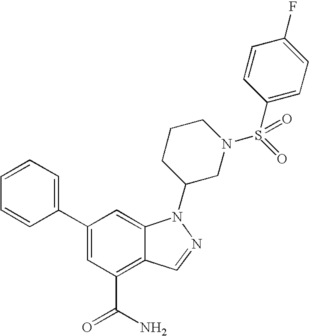

Following the general procedure of example 4, 6-phenyl-1-(3-piperidinyl)-1H-indazole-4-carboxamide (example 13) (61 mg, 0.171 mmol), triethylamine (95 uL, 0.68 mmol), DMAP (3.4 mg, 0.027 mmol) and 4-fluorobenzenesulphonyl chloride (33 mg, 0.171 mmol) were reacted to give the title compound (17.5 mg, 21%).

LC/MS: 478.00 Rt: 11.19 min.

### Biological Data

### IKK2 Assay

Recombinant human IKKβ (residues 1-737) was expressed in baculovirus as a C-terminal GST-tagged fusion protein, and its activity was assessed using a time-resolved fluorescence resonance energy transfer (TR-FRET) assay. Briefly, IKK2 (5 nM final) diluted in assay buffer (50 mM HEPES, 10 mM MgCl₂, 1 mM CHAPS pH 7.4 with 1 mM DTT and 0.01% w/v BSA) was added to wells containing various concentrations of compound or DMSO vehicle (3% final). The reaction was initiated by the addition of GST-IκBα substrate (25 nM final)/ATP (1 μM final), in a total volume of 30 μl. The reaction was incubated for 30 minutes at room temperature, then terminated by the addition of 15 μl of 50 mM EDTA. Detection reagent (15 μl) in buffer (100 mM HEPES pH 7.4, 150 mM NaCl and 0.1% w/v BSA) containing antiphosphoserine-IκBα-32/36 monoclonal antibody 12C2 (Cell Signalling Technology, Beverly Mass., USA) labelled with W-1024 europium chelate (Wallac O Y, Turku, Finland), and an APC-labelled anti-GST antibody (Prozyme, San Leandro, Calif., USA) was added and the reaction was further incubated for 60 minutes at room temperature. The degree of phosphorylation of GST-IκBα was measured using a Packard Discovery plate reader (Perkin-Elmer Life Sciences, Pangbourne, UK) as a ratio of specific 665 nm energy transfer signal to reference europium 620 nm signal.

### Results

The compounds of Examples 1-9, 12, and 14 were tested for activity against IKK2 and were found to be inhibitors of IKK2. These examples had a pIC₅₀ of 5.0 or greater. Examples 15-19 were also tested for activity against IKK2 and these compounds were found to have a pIC₅₀ less than 4.8.

### Monocyte Assay

Effect of IKK-β inhibition on human monocyte stimulated cytokine production was assessed as follows: Monocytes were isolated from heparinized whole blood by Ficoll gradient, followed by purification of CD14+ cells using MACS magnetic cell separation beads. Isolated monocytes were then adhered to 96-well culture plates at 1×10⁶ cells/mL in RPMI 1640 10% FBS (JRH Biosciences, Lenexa Kans.) for 2 h. to further enrich the monocyte population. The media was then removed, cells washed once with RPMI 1640, and 0.125 mL RPMI 1640 10% FBS was added to the wells. Test compounds are added to the wells 30 minutes prior to stimulation with a final vehicle concentration of 0.1% DMSO. Monocytes were activated by the addition of 200 ng/mL endotoxin (LPS; E. coli serotype 026:B6) (Sigma, St. Louis, Mo.) and incubated for 24 hrs at 37 C. Cell-free supernates were analyzed by ELISA for TNF-α using Pharmingen matched pair Abs. Viability of the cells was determined byl 0% trypan blue exclusion.

What is claimed is:

1. A compound according to formula (I): 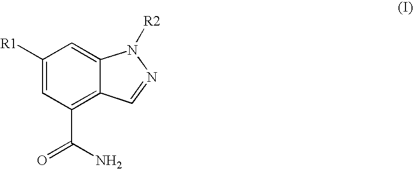
wherein:
R1 is optionally substituted aryl or optionally substituted heteroaryl,
where said aryl and heteroaryl are optionally substituted with one to three substituents each independently selected from the group consisting of: halo, optionally substituted C₁-C₆ alkyl, optionally substituted C₁-C₆ haloalkyl, —CN, —N(Rb)SO₂Re, —N(Rb)C(O)Ra, —C(O)NRaRb, —C(O)NRxRy, —SO₂NRaRb, —SO₂NRxRy, —ORc, —N(Rb)C(O)NRaRb, —N(Rb)C(O)NRxRy, and —N(Rb)C(O)ORd, where said C₁-C₆ alkyl and C₁-C₆ haloalkyl are optionally substituted with one to three substituents each independently selected from the group consisting of: NRaRb, —N(Rb)SO₂Re, C(O)Ra, —C(O)NRaRb, C₃-C₆ cycloalkyl, ORc, phenyl, and heterocycloalkyl optionally substituted with one or two C₁-C₆ alkyl groups;
R2 is H, or the group —YZ;
Y is a bond or C₁-C₆ alkylene;
Z is C₃-C₆ cycloalkyl, aryl, heteroaryl, or heterocycloalkyl each of which is optionally substituted by one R3 group;
R3 is R4, —S(O)₂R4, —C(O)R4, —C(O)OR4, —N(Rf)C(O)R4, —C(O)N(Rf)R4, —NHC(O)NHR4, —S(O)₂N(Rf)R4, or —N(Rf)S(O)₂R4;
R4 is optionally substituted C₁-C₆ alkyl, optionally substituted aryl, optionally substituted C₃-C₆ cycloalkyl, optionally substituted heteroaryl, or optionally substituted heterocycloalkyl,
wherein said C₁-C₆ alkyl is optionally substituted with one to three substituents each independently selected from the group consisting of: halo, —ORi, —NRgRh, —NHC(O)Rg, and Rj; and where said aryl and heteroaryl are optionally substituted by one to three substituents each independently selected from the group consisting of: halo, —ORg, nitro, cyano, —CF₃, C₁-C₆ alkyl, C(O)Rg, COORg, —NRgRh, —NHC(O)Rg, —C(O)NRgRh, —S(O)₂Rg, —NHS(O)₂Rg, and —S(O)₂NRgRh; and where said C₃-C₆ cycloalkyl and heterocycloalkyl are optionally substituted by one to three substituents each independently selected from the group consisting of: —OH, oxo, C₁-C₆ alkyl, and C₁-C₆ haloalkyl;
each Ra is independently selected from the group consisting of: H, optionally substituted C₁-C₃ alkyl, optionally substituted phenyl, optionally substituted heteroaryl, optionally substituted C₃-C₇ cycloalkyl, and optionally substituted heterocycloalkyl, where said C₁-C₃ alkyl is optionally substituted with one to three substituents each independently selected from the group consisting of: halo, ORc, C₁-C₆ haloalkyl, phenyl, and heteroaryl; and where said phenyl, heteroaryl, C₃-C₇ cycloalkyl, and heterocycloalkyl are optionally substituted with one to three substituents each independently selected from the group consisting of: halo, ORc, C₁-C₆ alkyl, and C₁-C₆ haloalkyl;
each Rb is independently selected from the group consisting of: H and optionally substituted C₁-C₃ alkyl, where said C₁-C₃ alkyl is optionally substituted with one to three ORc groups;
each Rc is independently selected from the group consisting of: H, optionally substituted C₁-C₆ alkyl, optionally substituted C₁-C₆ haloalkyl, optionally substituted C₃-C₇ cycloalkyl, optionally substituted heterocycloalkyl, optionally substituted aryl, optionally and substituted heteroaryl, where said C₁-C₆ alkyl and C₁-C₆ haloalkyl are optionally substituted with one to three substituents each independently selected from the group consisting of: C₃-C₆ cycloalkyl, phenyl, heterocycloalkyl, and heteroaryl; and where said aryl and heteroaryl are optionally substituted with one to three substituents each independently selected from the group consisting of: halo, C₁-C₃ alkyl, C₁-C₃ haloalkyl and OH; and where said C₃-C₇ cycloalkyl and heterocycloalkyl are optionally substituted with one to three C₁-C₃ alkyl groups;
each Rd is independently optionally substituted C₁-C₃ alkyl, where said C₁-C₃ alkyl is optionally substituted with one to three substituents each independently selected from the group consisting of: C₃-C₆ cycloalkyl; phenyl optionally substituted with one to three substituents each independently selected from the group consisting of: halo, C₁-C₆ alkyl, and C₃-C₆ cycloalkyl; and heteroaryl optionally substituted with one to three substituents each independently selected from the group consisting of: halo, C₁-C₆ alkyl, and C₃-C₆ cycloalkyl;
each Re is independently selected from the group consisting of: optionally substituted C₁-C₆ alkyl, optionally substituted phenyl, optionally substituted heteroaryl, optionally substituted C₅-C₇ cycloalkyl, and optionally substituted heterocycloalkyl, where said C₁-C₆ alkyl is optionally substituted with one substituent selected from the group consisting of: ORc, trifluoromethyl, phenyl, heteroaryl, heterocycloalkyl optionally substituted with ORc or heterocycloalkyl, and NRaRb; where said phenyl and heteroaryl are optionally substituted with one to three substituents each independently selected from the group consisting of: halo, CN, C₁-C₆ alkyl, C₁-C₆ haloalkyl, N(Rb)C(O)Ra, and ORf; and where said C₅-C₇ cycloalkyl and heterocycloalkyl are optionally substituted with one to three substituents each independently selected from the group consisting of: halo, C₁-C₆ alkyl optionally substituted with ORc and C₃-C₆ cycloalkyl;
each Rf is independently selected from the group consisting of: H and C₁-C₆ alkyl;
each Rg is independently selected from the group consisting of: H, C₁-C₆ alkyl, C₃-C₆ cycloalkyl, heteroaryl, and phenyl;
each Rh is independently selected from the group consisting of: H and C₁-C₆ alkyl optionally substituted with one phenyl group;
each Ri is independently selected from the group consisting of: H, C₁-C₆ alkyl, C₁-C₆ haloalkyl, and phenyl;
Rj is optionally substituted aryl, optionally substituted heteroaryl, optionally substituted C₃-C₆ cycloalkyl, or optionally substituted heterocycloalkyl,
wherein said aryl and heteroaryl are optionally substituted with one to three substituents each independently selected from the group consisting of: —ORf, nitro, cyano, CF₃, unsubstituted C₁-C₆ alkyl, —C(O)Rf, —COORf, —NRfRg, —NHC(O)Rf, —C(O)NRfRg, —S(O)₂Rf, —NHS(O)₂Rf, and —S(O)₂NRfRg; and where said C₃-C₆ cycloalkyl and heterocycloalkyl are optionally substituted with one to three substituents each independently selected from the group consisting of: —OH, oxo, C₁-C₆ alkyl, and C₁-C₆ haloalkyl; and
each Rx and Ry taken together with the nitrogen atom to which they are attached form a ring having from 5 to 7 member atoms wherein said ring optionally contains one additional heteroatom as a member atom, said ring is saturated or unsaturated but not aromatic, and said ring is optionally substituted with one or two C₁-C₃ alkyl substituents; or a pharmaceutically acceptable salt thereof.

2. A compound according to claim 1 wherein R1 is optionally substituted phenyl, or a pharmaceutically acceptable salt thereof.

3. A compound according to claim 1 wherein R1 is phenyl or a pharmaceutically acceptable salt thereof.

4. A compound according to claim 1 wherein Y is a bond or a pharmaceutically acceptable salt thereof.

5. A compound according to claim 1 wherein Z is a heterocycloalkyl group optionally substituted by one R3 group, or a pharmaceutically acceptable salt thereof.

6. A compound according to claim 1 wherein Z is piperidinyl optionally substituted by one R3 group or a pharmaceutically acceptable salt thereof.

7. A compound according to claim 1 wherein R3 is R4, —S(O)₂R4, —C(O)R4, or —C(O)OR4; or a pharmaceutically acceptable salt thereof.

8. A compound according to claim 1 wherein R4 is optionally substituted phenyl, optionally substituted heteroaryl, or optionally substituted C₁-C₆ alkyl; or a pharmaceutically acceptable salt thereof.

9. A compound according to claim 1 wherein R1 is phenyl; R2 is the group —YZ; Y is a bond; Z is piperidinyl substituted by one R3 group; R3 is R4, —S(O)₂R4, —C(O)R4, or —C(O)OR4; and R4 is C₁-C₆ alkyl, phenyl, 4-fluorophenyl, or 1-methyl-1H-imidazol-4-yl; or a pharmaceutically acceptable salt thereof.

10. A compound according to claim 1 selected from the group consisting of:
1,1-dimethylethyl-4-[4-(aminocarbonyl)-6-phenyl-1H-indazol-1-yl]-1-piperidinecarboxylate;
6-phenyl-1-(4-piperidinyl)-1H-indazole-4-carboxamide;
6-phenyl-1H-indazole-4-carboxamide;
1-{1-[(4-fluorophenyl)sulfonyl]-4-piperidinyl}-6-phenyl-1H-indazole-4-carboxamide;
1-{1-[(1-methyl-1H-imidazol-4-yl)sulfonyl]-4-piperidinyl}-6-phenyl-1H-indazole-4-carboxamide;
6-phenyl-1-(1-propanoyl-4-piperidinyl)-1H-indazole-4-carboxamide;
1-[1-(ethylsulfonyl)-4-piperidinyl]-6-phenyl-1H-indazole-4-carboxamide;
6-phenyl-1-[1-(phenylcarbonyl)-4-piperidinyl]-1H-indazole-4-carboxamide;
6-phenyl-1-[1-(phenylsulfonyl)-4-piperidinyl]-1H-indazole-4-carboxamide;
6-phenyl-1-[1-(phenylmethyl)-4-piperidinyl]-1H-indazole-4-carboxamide 1-(1-ethyl-4-piperidinyl)-6-phenyl-1H-indazole-4-carboxamide;
1,1-dimethylethyl-3-[4-(aminocarbonyl)-6-phenyl-1H-indazol-1-yl]-1-piperidinecarboxylate;
6-phenyl-1-(3-piperidinyl)-1H-indazole-4-carboxamide;
1-[1-(ethylsulfonyl)-3-piperidinyl]-6-phenyl-1H-indazole-4-carboxamide;
1-{1-[(1-methyl-1H-imidazol-4-yl)sulfonyl]-3-piperidinyl}-6-phenyl-1H-indazole-4-carboxamide;
6-phenyl-1-(1-propanoyl-3-piperidinyl)-1H-indazole-4-carboxamide;
6-phenyl-1-[1-(phenylcarbonyl)-3-piperidinyl]-1H-indazole-4-carboxamide;
6-phenyl-1-[1-(phenylsulfonyl)-3-piperidinyl]-1H-indazole-4-carboxamide; and
1-{1-[(4-fluorophenyl)sulfonyl]-3-piperidinyl}-6-phenyl-1H-indazole-4-carboxamide; or a pharmaceutically acceptable salt thereof.

11. A pharmaceutical composition comprising a compound according to claim 1, or a pharmaceutically acceptable salt thereof, and one or more of pharmaceutically acceptable excipients.

12. A method of treating a disorder mediated by inappropriate IKK2 activity comprising administering a safe and effective amount of a compound according to claim 1, or a pharmaceutically acceptable salt thereof, to a patient in need thereof.

13. A method according to claim 12 wherein the disorder mediated by inappropriate IKK2 activity is an inflammatory or tissue repair disorder.

14. A method according to claim 12 wherein the disorder mediated by inappropriate IKK2 activity is an autoimmune disease.

15. A method according to claim 14 wherein the autoimmune disease is systemic lupus eythematosus, multiple sclerosis, psoriatic arthritis, or alkylosing spondylitis.

16. A method according to claim 12 wherein the disorder mediated by inappropriate IKK2 activity is selected from the group consisting of: rheumatoid arthritis, inflammatory bowel disease, asthma, COPD (chronic obstructive pulmonary disease) osteoarthritis, osteoporosis, psoriasis, atopic dermatitis, ultraviolet radiation (UV)-induced skin damage, systemic lupus eythematosus, multiple sclerosis, psoriatic arthritis, alkylosing spondylitis, tissue rejection, organ rejection, Alzheimer's disease, stroke, atherosclerosis, restonosis, diabetes, glomerulonephritis, Hodgkins disease, cachexia, inflammation associated with infection and certain viral infections, including acquired immune deficiency syndrome (AIDS), adult respiratory distress syndrome, and Ataxia Telangiestasia.

17. A method according to claim 16 wherein the disorder mediated by inappropriate IKK2 activity is rheumatoid arthritis, asthma or COPD.

18. A method according to claim 17 wherein the disorder mediated by inappropriate IKK2 activity is rheumatoid arthritis.

19. A method according to claim 17 wherein the disorder mediated by inappropriate IKK2 activity is asthma.

20. A method according to claim 17 wherein the disorder mediated by inappropriate IKK2 activity is COPD.

21. A method according to claim 16 wherein the disorder mediated by inappropriate IKK2 activity is selected from the group consisting of: Alzheimer's disease, stroke atherosclerosis, restenosis, diabetes, glomerulonephritis, osteoarthritis, osteoporosis, and Ataxia Telangiestasia.

22. A method according to claim 12 wherein the disorder mediated by inappropriate IKK2 activity is cancer or cachexia.

23. A method according to claim 22 wherein the cancer is Hodgkin's disease.

24. An intermediate compound selected from:
1-(1-{[(1,1-dimethylethyl)oxy]carbonyl}-4-piperidinyl)-6-phenyl-1H-indazole-4-carboxylic acid;
methyl 1-(1-{[(11-dimethylethyl)oxy]carbonyl}-4-piperidinyl)-6-phenyl-1H-indazole-4-carboxylate;
methyl 6-phenyl-1H-indazole-4-carboxylate;
methyl 1-acetyl-6-phenyl-1H-indazole-4-carboxylate;
1-(1-{[(1,1-dimethylethyl)oxy]carbonyl}-3-piperidinyl)-6-phenyl-1H-indazole-4-carboxylic acid; and
methyl 1-(1-{[(1,1-dimethylethyl)oxy]carbonyl}-3-piperidinyl)-6-phenyl-1H-indazole-4-carboxylate.
